SEQUENCE LISTING (63 sequences)

SEQ ID NO: 1 - protein, 40 aa, artificial sequence
  Peptide compound
  MISC_FEATURE (1)..(6): Xaa  is absent or 1 amino acid
  MISC_FEATURE (9)..(9): Xaa  is 1 amino acid
  MISC_FEATURE (10)..(13): Xaa  is  absent or 1 amino acid
  MISC_FEATURE (16)..(16): Xaa is 1 amino acid
  MISC_FEATURE (17)..(20): Xaa is absent or 1 amino acid
  MISC_FEATURE (23)..(24): Xaa is 1 amino acid
  MISC_FEATURE (25)..(32): Xaa is absent or 1 amino acid
  MISC_FEATURE (35)..(40): Xaa is  absent or  1 amino acid
XXXXXXWSXXXXXWSXXXXXCSXXXXXXXXXXCGXXXXXX

SEQ ID NO: 2 - protein, 28 aa, Artificial sequence
  Peptide compound
  MISC_FEATURE (3)..(3): Xaa is 1 amino acid
  MISC_FEATURE (4)..(7): Xaa is absent or 1 amino acid
  MISC_FEATURE (10)..(10): Xaa is 1 amino acid
  MISC_FEATURE (11)..(14): Xaa is absent or 1 amino acid
  MISC_FEATURE (17)..(18): Xaa is 1 amino acid
  MISC_FEATURE (19)..(26): Xaa is absent or 1 amino acid
WSXXXXXWSXXXXXCSXXXXXXXXXXCG

SEQ ID NO: 3 - protein, 12 aa, Artificial sequence
  Peptide compound
WSGWSSCSRSCG

SEQ ID NO: 4 - protein, 12 aa, Artificial sequence
  Peptide compound
WSSWSGCSRSCG

SEQ ID NO: 5 - protein, 12 aa, artificial sequence
  peptide compound
WSSWGSCSRSCG

SEQ ID NO: 6 - protein, 12 aa, artificial sequence
  peptide compound
WSSWGGCSRSCG

SEQ ID NO: 7 - protein, 12 aa, artificial sequence
  peptide compound
WSSWSSCSRSCG

SEQ ID NO: 8 - protein, 12 aa, artificial sequence
  peptide compound
WSGWSGCSRSCG

SEQ ID NO: 9 - protein, 12 aa, artificial sequence
  peptide compound
WSGWGSCSRSCG

SEQ ID NO: 10 - protein, 12 aa, artificial sequence
  peptide compound
WSGWGGCSRSCG

SEQ ID NO: 11 - protein, 12 aa, artificial sequence
  peptide compound
WSSWSSCSVSCG

SEQ ID NO: 12 - protein, 12 aa, artificial sequence
  peptide compound
WSSWSGCSVSCG

SEQ ID NO: 13 - protein, 12 aa, artificial sequence
  peptide compound
WSSWGSCSVSCG

SEQ ID NO: 14 - protein, 12 aa, artificial sequence
  peptide compound
WSSWGGCSVSCG

SEQ ID NO: 15 - protein, 12 aa, artificial sequence
  peptide compound
WSGWSSCSVSCG

SEQ ID NO: 16 - protein, 12 aa, artificial sequence
  peptide compound
WSGWSGCSVSCG

SEQ ID NO: 17 - protein, 12 aa, artificial sequence
  peptide compound
WSGWGSCSVSCG

SEQ ID NO: 18 - protein, 12 aa, artificial sequence
  peptide compound
WSGWGGCSVSCG

SEQ ID NO: 19 - protein, 12 aa, artificial sequence
  peptide compound
WSSWSSCSVTCG

SEQ ID NO: 20 - protein, 12 aa, artificial sequence
  peptide compound
WSSWSGCSVTCG

SEQ ID NO: 21 - protein, 12 aa, artificial sequence
  peptide compound
WSSWGSCSVTCG

SEQ ID NO: 22 - protein, 12 aa, artificial sequence
  peptide compound
WSSWGGCSVTCG

SEQ ID NO: 23 - protein, 12 aa, artificial sequence
  peptide compound
WSGWSSCSVTCG

SEQ ID NO: 24 - protein, 12 aa, Artificial Sequence
  peptide compound
WSGWSGCSVTCG

SEQ ID NO: 25 - protein, 12 aa, Artificial Sequence
  peptide compound
WSGWGSCSVTCG

SEQ ID NO: 26 - protein, 12 aa, Artificial Sequence
  peptide compound
WSGWGGCSVTCG

SEQ ID NO: 27 - protein, 12 aa, Artificial Sequence
  peptide compound
WSSWSSCSRTCG

SEQ ID NO: 28 - protein, 12 aa, artificial sequence
  peptide compound
WSSWSGCSRTCG

SEQ ID NO: 29 - protein, 12 aa, Artificial Sequence
  peptide compound
WSSWGSCSRTCG

SEQ ID NO: 30 - protein, 12 aa, Artificial Sequence
  peptide compound
WSSWGGCSRTCG

SEQ ID NO: 31 - protein, 12 aa, Artificial Sequence
  peptide compound
WSGWSSCSRTCG

SEQ ID NO: 32 - protein, 12 aa, Artificial Sequence
  peptide compound
WSGWSGCSRTCG

SEQ ID NO: 33 - protein, 12 aa, Artificial Sequence
  peptide compound
WSGWGSCSRTCG

SEQ ID NO: 34 - protein, 12 aa, Artificial Sequence
  peptide compound
WSGWGGCSRTCG

SEQ ID NO: 35 - protein, 13 aa, Artificial Sequence
  peptide compound
PWSGWSSCSRSCG

SEQ ID NO: 36 - protein, 14 aa, Artificial Sequence
  peptide compound
APWSGWSSCSRSCG

SEQ ID NO: 37 - protein, 15 aa, Artificial Sequence
  peptide compound
LAPWSGWSSCSRSCG

SEQ ID NO: 38 - protein, 16 aa, Artificial Sequence
  peptide compound
VLAPWSGWSSCSRSCG

SEQ ID NO: 39 - protein, 13 aa, Artificial Sequence
  peptide compound
WSGWSSCSRSCGL

SEQ ID NO: 40 - protein, 14 aa, Artificial Sequence
  peptide compound
WSGWSSCSRSCGLG

SEQ ID NO: 41 - protein, 15 aa, Artificial Sequence
  peptide compound
WSGWSSCSRSCGLGL

SEQ ID NO: 42 - protein, 16 aa, Artificial Sequence
  peptide compound
WSGWSSCSRSCGLGLI

SEQ ID NO: 43 - protein, 17 aa, Artificial Sequence
  peptide compound
WSGWSSCSRSCGLGLIF

SEQ ID NO: 44 - protein, 14 aa, Artificial Sequence
  peptide compound
PWSGWSSCSRSCGL

SEQ ID NO: 45 - protein, 15 aa, Artificial Sequence
  peptide compound
PWSGWSSCSRSCGLG

SEQ ID NO: 46 - protein, 16 aa, Artificial Sequence
  peptide compound
PWSGWSSCSRSCGLGL

SEQ ID NO: 47 - protein, 17 aa, Artificial Sequence
  peptide compound
PWSGWSSCSRSCGLGLI

SEQ ID NO: 48 - protein, 18 aa, Artificial Sequence
  peptide compound
PWSGWSSCSRSCGLGLIF

SEQ ID NO: 49 - protein, 15 aa, Artificial Sequence
  peptide compound
APWSGWSSCSRSCGL

SEQ ID NO: 50 - protein, 16 aa, Artificial Sequence
  peptide compound
APWSGWSSCSRSCGLG

SEQ ID NO: 51 - protein, 17 aa, Artificial Sequence
  peptide compound
APWSGWSSCSRSCGLGL

SEQ ID NO: 52 - protein, 18 aa, Artificial Sequence
  peptide compound
APWSGWSSCSRSCGLGLI

SEQ ID NO: 53 - protein, 19 aa, Artificial Sequence
  peptide compound
APWSGWSSCSRSCGLGLIF

SEQ ID NO: 54 - protein, 16 aa, Artificial Sequence
  peptide compound
LAPWSGWSSCSRSCGL

SEQ ID NO: 55 - protein, 17 aa, Artificial Sequence
  peptide compound
LAPWSGWSSCSRSCGLG

SEQ ID NO: 56 - protein, 18 aa, Artificial Sequence
  peptide compound
LAPWSGWSSCSRSCGLGL

SEQ ID NO: 57 - protein, 19 aa, Artificial Sequence
  peptide compound
LAPWSGWSSCSRSCGLGLI

SEQ ID NO: 58 - protein, 20 aa, Artificial Sequence
  peptide compound
LAPWSGWSSCSRSCGLGLIF

SEQ ID NO: 59 - protein, 17 aa, Artificial Sequence
  peptide compound
VLAPWSGWSSCSRSCGL

SEQ ID NO: 60 - protein, 18 aa, Artificial Sequence
  peptide compound
VLAPWSGWSSCSRSCGLG

SEQ ID NO: 61 - protein, 19 aa, Artificial Sequence
  peptide compound
VLAPWSGWSSCSRSCGLGL

SEQ ID NO: 62 - protein, 20 aa, Artificial Sequence
  peptide compound
VLAPWSGWSSCSRSCGLGLI

SEQ ID NO: 63 - protein, 21 aa, Artificial Sequence
  peptide compound
VLAPWSGWSSCSRSCGLGLIF

(19) United States
(12) Patent Application Publication
(10) Pub. No.: US 2025/0177481 A1
(43) Pub. Date: Jun. 5, 2025

(54) SCO-spondin-derived polypeptides for enhancing synaptic transmission

(71) Applicant: AXOLTIS PHARMA, CLERMONT-FERRAND (FR)

(72) Inventors: Sighild Brunhilde Adeline LEMARCHANT, LYON (FR); Yann GODFRIN, CALUIRE (FR); Christine Mélissa SOURIOUX, LYON (FR)

(21) Appl. No.: 18/576,443

(22) PCT Filed: Jun. 17, 2022
(86) PCT No.: PCT/EP2022/066615

§ 371 Date: Jan. 4, 2024

(30) Foreign Application Priority Data
Jul. 9, 2021 (EP) 21305959.5
Dec. 2, 2021 (EP) 21306694.7

(51) Int. Cl. A61K 38/17; A61K 38/10; A61P 25/18; A61P 25/24

(52) U.S. Cl. CPC A61K 38/1709; A61K 38/10; A61P 25/18; A61P 25/24

(57) ABSTRACT

The invention relates to polypeptides derived from SCO-spondin for increasing or enhancing the basal excitatory synaptic transmission, notably glutamatergic neurotransmission. More particularly the invention relates to said polypeptides for increasing or enhancing glutamatergic neurotransmission in diseases or conditions comprising psychiatric disorders; drug addiction; viral infection (such as coronaviruses, e.g. SARS CoV2) related neurological symptoms; NMDA receptor (NMDAr) and/or AMPA receptor (AMPAr) deficiency related disease, notably anti-NMDAr encephalitis; vegetative state, and hypoxic brain injury. The present invention also relates to methods of treatment.

The present invention relates to polypeptides derived from SCO-spondin for increasing or enhancing the basal excitatory synaptic transmission, notably glutamatergic neurotransmission. More particularly the invention relates to said polypeptides for increasing or enhancing glutamatergic neurotransmission in diseases or conditions comprising psychiatric disorders; drug addiction; viral infection (such as coronaviruses, e.g. SARS CoV2) related neurological symptoms; NMDA receptor (NMDAr) and/or AMPA receptor (AMPAr) deficiency related disease, notably anti-NMDAr encephalitis; vegetative state and hypoxic brain injury. The present invention also relates to methods of treatment.

### BACKGROUND OF INVENTION

Disturbances in synapse physiology can result in major brain homeostasis defects that impair neural circuits and the execution of higher brain functions such as cognition and consciousness. Glutamate is the most abundant excitatory neurotransmitter in the human brain and plays a critical role in synaptic plasticity such as long-term potentiation (Hansen et al., 2017). Synapse-targeted therapies which selectively enhance NMDA- and/or AMPA-receptor-mediated glutamatergic activity in key cerebral circuits may improve brain activity in disorders or states where cognition or consciousness are altered such as psychiatric disorders, drug addiction, viral, especially coronavirus infections and their related neurological symptoms, NMDAr and/or AMPAr deficiency related disease, and vegetative state.

Impairment of glutamatergic neurotransmission is a common feature of many psychiatric disorders (Tang et al., 2020). Schizophrenia is a chronic debilitating psychiatric disease that affects ˜1% of the world's population. NMDA receptor hypofunction in schizophrenia is supported by clinical observations showing that administration of NMDA receptor antagonists (phencyclidine (PCP), ketamine) to normal healthy humans induces a spectrum of psychotic symptoms and cognitive impairments, that resemble to those exhibited by schizophrenic patients (Pratt et al., 2017; Hashimoto, 2014).

Hippocampal glutamatergic function is also altered in subjects with bipolar disorder for which a decrease in the number of NMDA receptors with open ion channels in certain regions of the hippocampus was observed (Scarr et al., 2003; Chitty et al., 2015).

SARS CoV2 long-haulers suffer from adverse neurological effects including long-lasting cognitive impairments (Kumar et al., 2021; Alnefeesi et al., 2020). Postmortem analyses of brain tissues from COVID-19 patients evidenced synaptic deficits in upper-layer excitatory neurons known to play a critical role in cognitive function (Yang et al., 2021). This neuronal population may therefore be particularly sensitive to deficits in neurotransmission with COVID-19 affected astrocytes and neurons.

Many drugs such as cocaine, amphetamine, opioids, alcohol and cannabis alter synaptic transmission and induce cognition-related symptoms (Gould, 2010). For example, chronic abuse of cocaine alters long term potentiation (Keralapurath et al., 2017) and produces deficits in cognitive flexibility (Jedema et al., 2021). Similarly, NMDA receptor antagonists (PCP and ketamine) when used as a drug or as an anesthetic restrain synaptic transmission and long-term potentiation in the hippocampus which induces cognitive impairments, notably working memory deficits (Roussy et al., 2021; Medina-Kirchner and Evans, 2021; Luo et al., 2002; Pratt et al., 2017; Hashimoto, 2014; Stringer et al., 1983). Withdrawal of opioids (morphine and heroin) alters synaptic function and long term-potentiation, leading to cognitive inflexibility (Gould, 2010; Pu et al., 2002).

Anti-NMDA receptor encephalitis is an autoimmune disease characterized by autoantibodies that target NMDAr present in the brain which substantially alters glutamatergic synaptic transmission (Wagnon et al., 2020; Tang et al., 2020; Finke et al., 2012). Anti-NMDAr encephalitis can be encountered in Coronavirus infection related diseases (Sarigecili et al., 2021; Alvarez Bravo and Ramio Torrenta, 2020) and in autoimmune autism (Tzang et al., 2019). This extent to diseases and conditions for which autoantibodies anti-NMDAr are detected such as stroke (Stanca et al., 2015), schizophrenia (Pearlman and Najjar, 2014), major depressive disorder, dementia (Doss et al., 2014), aging-related cognitive decline (Busse et al., 2014).

Vegetative state is defined as a strong reduction of the activity of neural circuits subtending consciousness resulting from traumatic and non-traumatic conditions. The thalamus plays a central role in the integration and transmission of neural information between the subcortical and cortical areas. Alterations of the synaptic strength and plasticity of thalamocortical projections represent major constraints for recovery of consciousness in patients in a vegetative state (Bagnato et al., 2013; Pistoia et al., 2010).

Hypoxia is a common condition in which some tissues of the body are starved of oxygen. Such lack of adequate oxygenation can have a dramatic impact on the entire affected tissue. Notably, an insufficient oxygen supply to the brain may cause a depression in synaptic transmission (usually referred as “hypoxia-induced depression of synaptic transmission”), whilst prolonged exposure to hypoxia leads to neuronal cell loss and death. If left unprevented or untreated, cerebral oxygen deprivation can thus result in hypoxic brain injury.

The disruption of synaptic function represents a major determinant of most neurodegenerative diseases, CNS injuries and psychiatric disorders. Disturbances in synapse physiology can unbalance brain homeostasis, thereby impairing the functional integrity of neural circuits and the execution of higher brain functions such as cognition and consciousness. Glutamate plays a critical role in synaptic plasticity such as long-term potentiation (LTP) [Hansen 2017]. Synapse-targeted therapies which selectively enhance α-amino-3-hydroxy-5-methyl-4-isoxazolepropionic acid receptor (AMPAR)- and/or N-methyl-D-aspartate receptor (NMDAR)-mediated glutamatergic transmission in key neuronal networks may therefore improve brain activity in disorders or states where cognition or consciousness are altered such as psychiatric disorders [Hashimoto 2014, Tang 2020, Balu 2016, Nazakawa 2020], drug addiction [Luo 2021], neurodegenerative diseases [Benitez 2021; Conway 2020; Milnerwood 2010; Lepeta 2016], viral infections (especially coronavirus infections and their related neurological symptoms) [Kumar 2021], anti-NMDAR encephalitis [Finke 2012], vegetative state [Bagnato 2013], or aging [Kumar 2019, Shi 2007].

Methods that could increase, enhance or restore the glutamatergic neurotransmission would be highly beneficial to patients enduring conditions associated with the therapeutic contexts described supra.

SCO-spondin-derived peptides have been described for their neuroprotective and neuroregenerative properties. The use of SCO-spondin-derived peptides for the treatment of spinal cord injury and tauopathies has been investigated in animal models. However, to date, no role has been suggested for their ability to improve glutamatergic synaptic transmission.

### SUMMARY OF THE INVENTION

This invention dealts with the effect of peptides on synaptic function/dysfunction. By using electrophysiology on mouse brain slices, we describe that NX218 potentiates excitatory postsynaptic currents through AMPAR and GluN2A-containing NMDAR (GluN2A-NMDAR), and increases basal synaptic transmission. Accordingly, a single acute systemic administration of NX218 in a mouse pharmacological model of synaptic dysfunction induced by the blockade of NMDAR with phencyclidine (PCP) improves spatial working memory. Further, repeated daily administrations of the peptide increase GluN2A-NMDAR protein levels and reverse PCP-induced decrease in NMDAR-driven signaling (phosphorylated cAMP response element-binding protein; pCREB), which also restores memory.

The inventors thus surprisingly identified a new property of SCO-spondin-derived peptides. The present invention relates to SCO-spondin-derived peptides or a pharmaceutical composition containing at least one of such peptides, for use in increasing or enhancing synaptic transmission, in particular the basal excitatory synaptic transmission, notably glutamatergic neurotransmission. As known and explained herein, impaired synaptic transmission, such as glutamatergic transmission, is a common feature of conditions or diseases, such as Schizophrenia, drug addiction, in particular generated by PCP, Ketamine or Scopolamine, NMDAr and/or AMPAr deficiency related disease, notably anti-NMDAr encephalitis, vegetative state, hypoxia-induced depression of synaptic transmission, hypoxic brain injury. These conditions or diseases are pathological conditions encompassed by said therapeutic use and corresponding methods of treatment.

This effect on synaptic transmission or neurotransmission may be obtained on existing fully functional synapses and/or on existing synapses, the function of which being impaired or inhibited. The peptide may increase, reestablish or protect the basal excitatory synaptic transmission, notably glutamatergic neurotransmission. As explained below, this is more particularly NMDAr related glutamatergic neurotransmission, especially linked to GluN2A subunit (GluN2A-NMDAR neurotransmission), and/or to AMPAR (AMPAR neurotransmission). More precisly, the effect is an increase or a reinforcement of the strength of neurotransmission in different neural circuits (i.e., hippocampal or thalamocortical synapses), through increases in GluN2A-NMDAR and AMPAR excitatory postsynaptic currents.

In an aspect, the peptides induce an increase or enhancing of the basal excitatory synaptic transmission, i.e. of the propagation of the signal from the presynaptic neuron to the postsynaptic one. The postsynaptic responses evoked by the electrical stimulation of the presynaptic terminals are thus increased when administering the peptide.

In an aspect, the peptides induce more specifically an increase, enhancing or restoration of glutamatergic neurotransmission. The glutamatergic responses recorded in the postsynaptic neurons are thus increased by the peptide application, with respect to the response recorded in the same neurons before the peptide application. In particular, there is an increase of the amplitude of these postsynaptic currents mediated by glutamate receptors, as illustrated in the examples with NMDAr electrical postsynaptic current (EPSC) amplitude: 101 pA in presence of the peptide versus 53 pA prior to peptide exposure; AMPAr EPSC amplitude: 163 pA in presence of peptide versus 142 pA prior to peptide exposure. This increased synaptic input may allow restoring action potential generation in the postsynaptic neurons. This increased synaptic input will make the postsynaptic neuron more likely to fire action potentials.

In an aspect, the increase or enhancing of NMDAr-related neurotransmission by the peptides is at least specifically induced by GluN2A subunit. When GluN2A current is isolated using a GluN2B subunit antagonist, it is shown that the addition of the peptides significantly increases the amplitude of NMDAr currents (GluN2A EPSC amplitude (% of control): 79% with GluN2B antagonist, and 91% with GluN2B antagonist and the peptide, see examples). Therefore, GluN2A subunit is involved in the increase of NMDAr currents amplitude mediated by the peptide. On the contrary, when GluN2B current is isolated using a GluN2A subunit antagonist, it is shown that the addition of the peptides has no significant effect on NMDAr currents. Therefore, GluN2B subunit is not involved in the increase of NMDAr currents amplitude mediated by the peptide. Additionally, in silico docking approaches confirm the binding of the peptides specifically on the GluN2A subunit of NMDAr, and on AMPA receptors.

Neurotransmission or synaptic transmission is the process by which one neuron chemically communicates with another that encodes information under the form of an electrical impulse called action potential. Once the action potential reaches the end of the axon of the neuron it needs to be transferred to another neuron or tissue. To achieve this, it must cross the synaptic gap between the presynaptic neuron and the postsynaptic neuron. At the end of the presynaptic neuron axon terminal are the synaptic vesicles, which contain chemical messengers, known as neurotransmitters. When the presynaptic action potential reaches these synaptic vesicles, they release their contents of neurotransmitters. Neurotransmitters then carry the signal across the synaptic gap. They bind to receptor sites on the postsynaptic neuron, thereby completing the process of synaptic transmission leading to an excitatory postsynaptic potential (EPSP) (i.e. the depolarization of the postsynaptic membrane) and possibly to the emission of a postsynaptic action potential. Among them, glutamate is the most abundant excitatory neurotransmitter in the human brain, therefore glutamatergic neurotransmission plays a key role in brain activity.

Using electrophysiological methods, it is possible to highlight any alteration or potentiation of the synaptic transmission, notably glutamatergic neurotransmission. This can be done by measuring any change in synaptic strength at glutamatergic synapses, through the recording of the postsynaptic current evoked in response to electrical presynaptic stimulation (Pons-Bennacoeur and Lozovaya, 2017).

By “increasing or enhancing glutamatergic neurotransmission” it is meant that the glutamatergic postsynaptic current would be of higher amplitude than expected (or than recorded in control conditions) when the neurons or the synapses are in presence of the peptides of the invention.

NMDAr postsynaptic current amplitude expressed in picoamperes (pA) may in particular increase of at least about 10 to 300% following peptide administration with respect to pre-treatment status or control condition. Preferably, the increase is between about 20 and about 40% for the AMPA receptor-mediated currents and between about 60 and about 230% for the NMDA receptor-mediated currents.

Current amplitude (pA) may in practice be measured using electrophysiology on brain slices from adult mice, based on known methods, such as those disclosed in the examples 4 and 5.

In an aspect of the invention, the peptides or pharmaceutical compositions are for use in preventing or treating a disease or condition which comprises psychiatric disorders, drug addiction, NMDAr and/or AMPAr deficiency related disease, notably anti-NMDAr encephalitis, vegetative state and hypoxic brain injury. In particular, the peptides are beneficial to the subject suffering or in risk of suffering of one of these diseases, through an increase, enhancing or restoration of glutamatergic neurotransmission, as disclosed herein. It is also beneficial to a normal subject for which an increase of said neurotransmission is wished. The peptides thus allow for an improvement (e.g. increase, enhancing or restoring) of the glutamatergic neurotransmission and thus an improvement of the functional or intellectual faculties involving the synapses for which the peptides increase, reestablish or protect the glutamatergic neurotransmission.

In an aspect, the invention is the use of SCO-spondin-derived peptides or a pharmaceutical composition containing at least one of such peptides, for preventing or treating the deleterious effect of hypoxia on synaptic transmission (depression of synaptic transmission represented by a substantial decrease in fEPSP slope), especially in the hippocampus. Cerebral hypoxia may be hypoxia occurring in the course of, or at the onset of, diseases, such as ischemic stroke, transient ischemic attack or any other condition resulting in cerebral ischemia, traumatic brain injuries, cardiac arrest or other heart problems, lung diseases (such as chronic obstructive pulmonary disease, emphysema, bronchitis, pneumonia, and pulmonary edema), perinatal hypoxic-ischemic encephalopathy (HIE), severe asthma attack, obstructive sleep apnea, obesity hypoventilation syndrome (OHS), anemia, infectious respiratory diseases (such as COVID-19 syndrome) and more generally any acute or chronic respiratory failure leading to prolonged or recurrent hypoventilation and any condition resulting in an inadequate oxygen delivery to the brain.

The peptide may in particular allow keeping normal synaptic function or close to normal, and/or recovering normal synaptic function or increasing synaptic transmission.

In an aspect, the peptide is used to promote excitatory postsynaptic potentials (EPSPs), especially promotes recovery of EPSPs from depression. As presented in examples 7 and 8, such an effect has been measured on hippocampal slices and through the measurement of field EPSP slopes in accordance with established method.

In an aspect, the peptide is used to preserve and/or rescue synaptic transmission (as shown by a recovery of the field excitatory postsynaptic potentials slope) when/if compromised.

In an aspect, the peptide is used to promote a better and faster recovery of the synaptic transmission when/if compromised.

In an aspect, the peptides may be beneficial to prevent the deleterious effects of hypoxia in the brain, especially the deleterious effects hypoxia may induce on the synaptic transmission, such as in the hippocampus, and/or to allow recovering normal synaptic transmission during hypoxia or after hypoxia.

It is demonstrated herein that a peptide as disclosed herein, e.g. NX218 triggered synaptic transmission through GluN2A-NMDAR and AMPAR, and even NMDAR-driven signaling as shown by the increase in pCREB cerebral content after repeated administrations of the peptide in vivo.

In an aspect, the peptide is used to treat or prevent a synaptopathy.

A peptide as disclosed herein, e.g. NX218 facilitates AMPAR- and GluN2A-NMDAR-mediated neurotransmission in brain areas associated with high-order functions (i.e., cortex and hippocampus). The treatment with such peptide elicits favorable changes both in NMDAR-dependent signaling and in short-term memory, as demonstrated in a pharmacological mouse model of synaptic dysfunction. Overall, the regulation of GluN2A-NMDAR and AMPAR function by said peptide represents a therapeutic opportunity to ameliorate outcomes in the elderly and in patients suffering from CNS disorders with disabling synaptic defects or synaptopathy.

### DETAILED DESCRIPTION

### Synaptic Dysfunction Detection:

Nowadays, several techniques are available to assess synaptic dysfunction in humans. Some electrophysiological measurements, imaging techniques and fluid biomarkers testing can notably allow diagnosis of synaptic dysfunction in patients in a non-invasive manner. The oldest and best-known method is electroencephalography (EEG), an electrophysiological monitoring method to record the electrical activity produced by the neurons in the brain (Cook et al., 1996). This electrical activity is represented by different EEG frequency bands (referred to as alpha, beta, gamma, theta and delta waves). Human EEG waves are well-characterized by different parameters values (main frequency, voltage and morphology). This allows medical professionals to detect quickly and easily any abnormal brain activity.

To study more specifically the activity within a particular central nervous system pathway, evoked potentials (EPs) can be even more useful in clinical medicine. EPs are used to measure the electrical activity in certain areas of the brain in response to an external stimulation, whether visual, auditory, sensitive or motor. The delay between the stimulation and the recorded electrical response as well as its amplitude are compared to the values usually obtained in healthy subjects. EPs are therefore very useful to know if a specific nervous system pathway is not functioning normally.

EPs can be used in addition to magnetic resonance imaging (MRI): MRI will detect possible lesions, while EPs will provide information on the functional impact of these lesions. More importantly, EPs allow to diagnose a dysfunction in the absence of any radiological abnormality.

More recently, some imaging techniques have also been customized to study synaptic function. This is the case for positron emission tomography (PET) imaging, a quantitative imaging technique, suitable to provide functional and physiological information in the whole living body. Regarding the brain, novel tracers have been developed to quantify synaptic function in human brain. The most used to date is the [11C]UCB-J PET, a radiotracer that binds to the presynaptic vesicle, thus allowing to detect a loss of connectivity across the brain and/or to track changes in synaptic function (Finnema et al., 2016).

Some CSF biomarkers can also be dosed to evaluate synaptic function. In the last decade, major advances in protein detection methods made it possible to accurately quantify pre- and postsynaptic proteins in biological fluids. As of today, the main synaptic biomarkers used for the study of synaptic function are the growth-associated protein 43 (GAP-43), the synaptosomal-associated protein 25 (SNAP-25), the synaptotagmin-1 and the neurogranin (Camporesi et al., 2020). Interestingly, many other biomarkers are currently emerging.

### SCO-Spondin Derived Peptides for Use in the Uses and Methods Disclosed Herein:

“SCO-Spondin” is a glycoprotein specific to the central nervous system and present in all of the vertebrates, from prochordals to humans. It is known as a molecule of extracellular matrices that is secreted by a specific organ located in the roof of the third ventricle, the sub-commissural organ. It is a molecule of large size. It consists of more than 4,500 amino acids and has a multi-modular organization that comprises various preserved protein patterns, including in particular 26 TR or TSR patterns. It is known that certain peptides derived from SCO-Spondin starting from TSR patterns have a biological activity in the nerve or neural cells (in particular described in WO-99/03890).

“TSR or TR patterns” are protein domains of approximately 55-60 residues, based on the alignment of preserved amino acids cysteine, tryptophan and arginine. These patterns were first isolated in TSP-1 (thrombospondin 1), a molecule that intervenes in coagulation. They were then described in numerous other molecules such as SCO-Spondin. In fact, this thrombospondin type 1 unit (TSR) comprises, in all the proteins studied so far and previously mentioned, about 55-60 amino acids (AA) some of which, like cysteine (C), tryptophan (W), serine (S), glycine (G), arginine (R) and proline (P) are highly conserved.

SCO-Spondin peptides or peptide compounds are used in performing the invention (the different objects of the invention, say peptide or composition for use, method of use, method of treatment, use of a peptide for the manufacture of a medicament, etc.). Also used are pharmaceutical compositions comprising at least one of the peptides according to the invention, and a pharmaceutically acceptable vehicle, carrier or diluent.

In particular, the invention uses a peptide of sequence
X1-W-S-A1-W-S-A2-C-S-A3-A4-C-G-X2 (SEQ ID NO: 1)
in which:
A1, A2, A3 and A4 consists of amino acid sequences consisting of 1 to 5 amino acids, the two cysteines form a disulfide bridge or not,
X1 and X2 consists of amino acid sequences consisting of 1 to 6 amino acids; or X1 and X2 are absent;
it being possible for the N-terminal amino acid to be acetylated (e.g. bears H₃CCOHN—), for the C-terminal amino acid to be amidated (e.g. bears —CONH₂), or both the N-terminal amino acid to be acetylated and the C-terminal amino acid to be amidated.

In an embodiment, in the formula of SEQ ID NO: 1, X1 or X2 or both X1 and X2 are absent. In an embodiment, where X1 and/or X2 is absent, the N-terminal W is acetylated and/or the C-terminal G is amidated. Preferably, both X1 and X2 are absent and the N-terminal W is acetylated and the C-terminal G is amidated.

In particular, the invention uses a peptide of sequence
W-S-A1-W-S-A2-C-S-A3-A4-C-G (SEQ ID NO: 2)
in which:
A1, A2, A3 and A4 consists of amino acid sequences consisting of 1 to 5 amino acids, the two cysteines form a disulfide bridge or not.

In an embodiment of the formulae of SEQ ID NO: 1 and 2, the peptide is a linear peptide, or the cysteines appearing on the peptide formula of SEQ ID NO: 1 and 2 do not form a disulfide bridge (reduced form).

In a preferred embodiment, the two cysteines appearing on the peptide formula of SEQ ID NO: 1 and 2 form a disulfide bridge (oxidized form).

Preferably, in the formulae of SEQ ID NO: 1 and 2, A1, A2, A3 and/or, preferably and A4 consist preferably of 1 or 2 amino acids, more preferably of 1 amino acid.

Preferably, A1 is chosen from G, V, S, P and A, more preferably G, S.

Preferably A2 is chosen from G, V, S, P and A, more preferably G, S.

Preferably, A3 is chosen from R, A and V, more preferably R, V.

Preferably, A4 is chosen from S, T, P and A, more preferably S, T.

Preferably, A1 and A2 are independently chosen from G and S.

Preferably, A3-A4 is chosen from R-S or V-S or V-T or R-T.

Preferably, X1, X2, A1, A2, A3 and A4 do not comprise cysteine.

When X1 is an amino acid sequence of 1 to 6 amino acids, the amino acids are any amino acid, and preferably chosen from V, L, A, P, and any combination thereof.

When X2 is an amino acid sequence of 1 to 6 amino acids, the amino acids are any amino acid, and preferably chosen from L, G, I, F, and any combination thereof.

In an embodiment, the peptide of SEQ ID NO: 1 or 2 is such that A1 and A2 are independently chosen from G and S and A3-A4 is chosen from R-S or V-S or V-T or R-T.

In a particular modality, this peptide is further acetylated and/or amidated. In an embodiment, the peptide is a linear peptide, or the cysteines do not form a disulfide bridge. In another embodiment, the peptide has the two cysteines forming a disulfide bridge (C-terminal cyclization). In another embodiment, the peptide as used in the invention or the peptide administered to the patient does comprise both forms, oxidized peptide and linear peptide.

For the purposes of the present invention, the term “amino acids” means both natural amino acids and non-natural amino acids and changes of amino acids, including from natural to non-natural, may be made routinely by the skilled person while keeping the function or efficacy of the original peptide. By “natural amino acids” is meant the amino acids in L form that may be found in natural proteins, i.e. alanine, arginine, asparagine, aspartic acid, cysteine; glutamine, glutamic acid, glycine, histidine, isoleucine, leucine, lysine, methionine, phenylalanine, proline, serine, threonine, tryptophan, tyrosine and valine. By “non-natural amino acid” is meant the preceding amino acids in their D form, as well as the homo forms of certain amino acids such as arginine, lysine, phenylalanine and serine, or the nor forms of leucine or valine. This definition also comprises other amino acids such as alpha-aminobutyric acid, agmatine, alpha-aminoisobutyric acid, sarcosine, statin, ornithine, deaminotyrosine. The nomenclature used to describe the peptide sequences is the international nomenclature using the one-letter code and where the amino-terminal end is shown on the left and the carboxy-terminus is shown on the right. The dashes “-” represent common peptide bonds linking the amino acids of the sequences.

In an embodiment, the peptide according to the invention, for example any one of the peptides of sequence SEQ ID NO: 1-63, comprises an N-terminal acetylation, a C-terminal amidation, or both an N-terminal acetylation and a C-terminal amidation.

In different embodiments, the invention relates to the use of polypeptides consisting essentially of, or consisting of the following amino acid sequences (Table A):

| | |
|---|---|
| W-S-G-W-S-S-C-S-R-S-C-G | SEQ ID NO: 3 |
| W-S-S-W-S-G-C-S-R-S-C-G | SEQ ID NO: 4 |
| W-S-S-W-G-S-C-S-R-S-C-G | SEQ ID NO: 5 |
| W-S-S-W-G-G-C-S-R-S-C-G | SEQ ID NO: 6 |
| W-S-S-W-S-S-C-S-R-S-C-G | SEQ ID NO: 7 |
| W-S-G-W-S-G-C-S-R-S-C-G | SEQ ID NO: 8 |
| W-S-G-W-G-S-C-S-R-S-C-G | SEQ ID NO: 9 |
| W-S-G-W-G-G-C-S-R-S-C-G | SEQ ID NO: 10 |
| W-S-S-W-S-S-C-S-V-S-C-G | SEQ ID NO: 11 |
| W-S-S-W-S-G-C-S-V-S-C-G | SEQ ID NO: 12 |
| W-S-S-W-G-S-C-S-V-S-C-G | SEQ ID NO: 13 |
| W-S-S-W-G-G-C-S-V-S-C-G | SEQ ID NO: 14 |
| W-S-G-W-S-S-C-S-V-S-C-G | SEQ ID NO: 15 |
| W-S-G-W-S-G-C-S-V-S-C-G | SEQ ID NO: 16 |
| W-S-G-W-G-S-C-S-V-S-C-G | SEQ ID NO: 17 |
| W-S-G-W-G-G-C-S-V-S-C-G | SEQ ID NO: 18 |
| W-S-S-W-S-S-C-S-V-T-C-G | SEQ ID NO: 19 |
| W-S-S-W-S-G-C-S-V-T-C-G | SEQ ID NO: 20 |
| W-S-S-W-G-S-C-S-V-T-C-G | SEQ ID NO: 21 |
| W-S-S-W-G-G-C-S-V-T-C-G | SEQ ID NO: 22 |
| W-S-G-W-S-S-C-S-V-T-C-G | SEQ ID NO: 23 |
| W-S-G-W-S-G-C-S-V-T-C-G | SEQ ID NO: 24 |
| W-S-G-W-G-S-C-S-V-T-C-G | SEQ ID NO: 25 |
| W-S-G-W-G-G-C-S-V-T-C-G | SEQ ID NO: 26 |
| W-S-S-W-S-S-C-S-R-T-C-G | SEQ ID NO: 27 |
| W-S-S-W-S-G-C-S-R-T-C-G | SEQ ID NO: 28 |
| W-S-S-W-G-S-C-S-R-T-C-G | SEQ ID NO: 29 |
| W-S-S-W-G-G-C-S-R-T-C-G | SEQ ID NO: 30 |
| W-S-G-W-S-S-C-S-R-T-C-G | SEQ ID NO: 31 |
| W-S-G-W-S-G-C-S-R-T-C-G | SEQ ID NO: 32 |
| W-S-G-W-G-S-C-S-R-T-C-G | SEQ ID NO: 33 |
| W-S-G-W-G-G-C-S-R-T-C-G | SEQ ID NO: 34 |

In an embodiment, the peptides of sequences SEQ ID NO: 3-34 disclosed in Table A are linear peptides, or the cysteines do not form a disulfide bridge (reduced peptides). In another embodiment, the peptides of sequences SEQ ID NO: 3-34 disclosed in the preceding Table A have the two cysteines oxidized to form a disulfide bridge (oxidized peptides). In another embodiment, the peptides as used in the invention or the peptides administered to the patient do comprise both forms, oxidized peptide and linear peptide of the same peptide sequence. In still another embodiment, the peptides as used in the invention or the peptides administered to the patient do comprise a mixture of at least two of these different peptides chosen from sequences SEQ ID NO: 3-34, wherein the mixture may be a mixture of at least two linear peptides or a mixture of at least two oxidized peptides, or a mixture of at least one linear peptide and at least one oxidized peptide, for example having the same amino acid sequence.

In a preferred embodiment, the peptide consists of the amino acid sequence W-S-G-W-S-S-C-S-R-S-C-G (SEQ ID NO: 3). In an embodiment, the peptide is a linear peptide, or the cysteines do not form a disulfide bridge (reduced form called NX210). In another embodiment, the peptides have the two cysteines oxidized to form a disulfide bridge (oxidized form), it is called NX218. In another embodiment, the peptides as used in the invention or the peptides administered to the patient do comprise both forms, oxidized and reduced.

In an embodiment of the peptides of SEQ ID NO: 1,
X1 represents a hydrogen atom or P or A-P or L-A-P or V-L-A-P, and/or
X2 represents a hydrogen atom or L or L-G or L-G-L or L-G-L-I or L-G-L-I-F.

In different embodiments, the invention thus relates to the use of polypeptides consisting or consisting essentially of the following amino acid sequences (Table B):

| | |
|---|---|
| P-W-S-G-W-S-S-C-S-R-S-C-G | SEQ ID NO: 35 |
| A-P-W-S-G-W-S-S-C-S-R-S-C-G | SEQ ID NO: 36 |
| L-A-P-W-S-G-W-S-S-C-S-R-S-C-G | SEQ ID NO: 37 |
| V-L-A-P-W-S-G-W-S-S-C-S-R-S-C-G | SEQ ID NO: 38 |
| W-S-G-W-S-S-C-S-R-S-C-G-L | SEQ ID NO: 39 |
| W-S-G-W-S-S-C-S-R-S-C-G-L-G | SEQ ID NO: 40 |
| W-S-G-W-S-S-C-S-R-S-C-G-L-G-L | SEQ ID NO: 41 |
| W-S-G-W-S-S-C-S-R-S-C-G-L-G-L-I | SEQ ID NO: 42 |
| W-S-G-W-S-S-C-S-R-S-C-G-L-G-L-I-F | SEQ ID NO: 43 |
| P-W-S-G-W-S-S-C-S-R-S-C-G-L | SEQ ID NO: 44 |
| P-W-S-G-W-S-S-C-S-R-S-C-G-L-G | SEQ ID NO: 45 |
| P-W-S-G-W-S-S-C-S-R-S-C-G-L-G-L | SEQ ID NO: 46 |
| P-W-S-G-W-S-S-C-S-R-S-C-G-L-G-L-I | SEQ ID NO: 47 |
| P-W-S-G-W-S-S-C-S-R-S-C-G-L-G-L-I-F | SEQ ID NO: 48 |
| A-P-W-S-G-W-S-S-C-S-R-S-C-G-L | SEQ ID NO: 49 |
| A-P-W-S-G-W-S-S-C-S-R-S-C-G-L-G | SEQ ID NO: 50 |
| A-P-W-S-G-W-S-S-C-S-R-S-C-G-L-G-L | SEQ ID NO: 51 |
| A-P-W-S-G-W-S-S-C-S-R-S-C-G-L-G-L-I | SEQ ID NO: 52 |
| A-P-W-S-G-W-S-S-C-S-R-S-C-G-L-G-L-I-F | SEQ ID NO: 53 |
| L-A-P-W-S-G-W-S-S-C-S-R-S-C-G-L | SEQ ID NO: 54 |
| L-A-P-W-S-G-W-S-S-C-S-R-S-C-G-L-G | SEQ ID NO: 55 |
| L-A-P-W-S-G-W-S-S-C-S-R-S-C-G-L-G-L | SEQ ID NO: 56 |
| L-A-P-W-S-G-W-S-S-C-S-R-S-C-G-L-G-L-I | SEQ ID NO: 57 |
| L-A-P-W-S-G-W-S-S-C-S-R-S-C-G-L-G-L-I-F | SEQ ID NO: 58 |
| V-L-A-P-W-S-G-W-S-S-C-S-R-S-C-G-L | SEQ ID NO: 59 |
| V-L-A-P-W-S-G-W-S-S-C-S-R-S-C-G-L-G | SEQ ID NO: 60 |
| V-L-A-P-W-S-G-W-S-S-C-S-R-S-C-G-L-G-L | SEQ ID NO: 61 |
| V-L-A-P-W-S-G-W-S-S-C-S-R-S-C-G-L-G-L-I | SEQ ID NO: 62 |
| V-L-A-P-W-S-G-W-S-S-C-S-R-S-C-G-L-G-L-I-F | SEQ ID NO: 63 |

In an embodiment, the peptides of sequences SEQ ID NO: 35-63 disclosed in Table B, or of sequences SEQ ID NO: 3-63 disclosed in Tables A+B, are linear peptides, or the cysteines do not form a disulfide bridge (reduced peptides). In another embodiment, the peptides have the two cysteines oxidized to form a disulfide bridge (oxidized peptides). In another embodiment, the peptides as used in the invention or the peptides administered to the patient do comprise both forms, oxidized peptide and linear peptide of the same peptide sequence. In still another embodiment, the peptides as used in the invention or the peptides administered to the patient do comprise a mixture of at least two of these different peptides chosen from sequences SEQ ID NO: 35-63, or 3-63, wherein the mixture may be a mixture of at least two linear peptides or a mixture of at least two oxidized peptides, or a mixture of at least one linear peptide and at least one oxidized peptide, for example having the same amino acid sequence.

Each one of the peptides of sequences SEQ ID NO: 3-63 may be acetylated, amidated, or acetylated and amidated.

The peptides as used in the invention or the peptides administered to the patient are defined with their amino acid sequences. The peptides as used may be one peptide as disclosed herein, or a mixture of at least two peptides as disclosed herein. The mixtures also encompass the mixture of linear and oxidized peptides, of the same or different amino acid sequences. If a 100% pure peptide may be used, in accordance with the invention, it is possible, and the invention encompasses, that the peptide has a purity greater than 80%, preferably 85%, more preferably 90%, even more preferably equal to or greater than 95, 96, 97, 98, or 99%. Conventional purification methods, for example by chromatography, may be used to purify the desired peptide compound.

In an embodiment, the peptide as used in the invention or the peptide administered to the patient do comprise both forms, oxidized peptide (Op) and linear peptide (Lp), for instance in similar amounts or not, e.g. (% in number) Op: 10, 20, 25, 30, 40, 50, 60, 70, 80, or 90%, the remaining to 100% being the Lp. The oxidized peptide and the linear peptide that are combined may be of the same sequence or of different sequences. For example, the oxidized and linear forms of the peptide of sequence SEQ ID NO: 3 are so combined (NX210 and NX218), for example in the proportions disclosed above. The same apply to any one of the peptides of sequence SEQ ID NO: 4-34 and 35-63.

In a general aspect, the peptides and pharmaceutical compositions of the invention are for use in enhancing or restoring excitatory synaptic transmission.

In an aspect, the peptides and pharmaceutical compositions are for use in enhancing basal excitatory synaptic transmission, in particular glutamatergic neurotransmission, more particularly NMDAr related glutamatergic neurotransmission, especially linked to GluN2A subunit.

In another aspect, the peptides and pharmaceutical compositions are for use in enhancing or restoring excitatory synaptic transmission when/if compromised, in particular during or after hypoxia.

In specific aspects, the peptides and pharmaceutical compositions are:
for use in treating or preventing Schizophrenia,
for use in treating or preventing bipolar disorder,
for use in treating or preventing drug addiction, in particular generated by PCP, Ketamine or Scopolamine,
for preventing or treating a NMDAr and/or AMPAr deficiency related disease, notably anti-NMDAr encephalitis,
for use in treating vegetative state,
for use in preventing and/or treating hypoxia-induced depression of synaptic transmission,
for use in preventing and/or treating hypoxic brain injury,
for use in treating or preventing synaptic deficits resulting from a viral infection, especially in SARS CoV2 and in COVID-19 (particularly long-haulers sick persons),
for use in increasing, enhancing or restoring excitatory synaptic transmission
for use in preventing or treating the deleterious effect of hypoxia on excitatory synaptic transmission,
for use in increasing, enhancing or restoring excitatory synaptic transmission, especially in the hippocampus, or for preventing or treating the deleterious effect of hypoxia on excitatory synaptic transmission, especially at the hippocampus,
for use in increasing NMDAr and/or AMPAr electrical postsynaptic current (EPSC), especially EPSC amplitude,
and combinations thereof.

### Schizophrenia and Drug Addiction:

In an aspect, the peptides or pharmaceutical compositions are used for preventing or treating Schizophrenia. In particular, the peptides are beneficial through an increase or enhancing of glutamatergic neurotransmission, as disclosed herein. The peptide increases, reestablishes or protects the glutamatergic neurotransmission. As disclosed herein, PCP and Scopolamine animal models are useful models relevant for Schizophrenia.

In an aspect, the peptides or pharmaceutical compositions are used for preventing or treating Drug addiction, for example Drug addiction generated by PCP or Ketamine or Scopolamine. In particular, the peptides are beneficial through an increase or enhancing of glutamatergic neurotransmission, as disclosed herein. The peptide increases, reestablishes or protects the glutamatergic neurotransmission.

Drug substances which are known to considerably alter synaptic transmission by antagonizing NMDA and/or AMPA and Acetylcholine (ACh) receptors, may benefit from the treatment with the peptides disclosed herein. Increasing or enhancing glutamatergic neurotransmission can play a key role to counteract cognitive deficits that can be induced by these Drug substances. These Drug substances comprise psychoactive substances such as PCP and Ketamine which are NMDAr antagonists and anticholinergic agents also known as tropane alkaloids such as Scopolamine.

Pharmacological blockade of glutamatergic or cholinergic synaptic transmission by PCP or Scopolamine, respectively, are also standard approaches to test putative cognitive enhancers and psychoactive compounds.

PCP model is of relevance for Schizophrenia and Drug addiction. Administration of the NMDA receptor antagonist, phencyclidine (PCP), produces schizophrenia-like symptoms in healthy volunteers (Domino and Luby, 2012) and is therefore often used to mimic schizophrenia in rodents (Jones et al., 2011). Symptoms of acute as well as subchronic administration of PCP in rodents include positive and negative symptoms (hyperlocomotion and social withdrawal, respectively), prepulse inhibition deficits and cognitive impairments (Young et al., 2012; Jones et al., 2011). The blockade of NMDA receptor-dependent current with antagonists such as PCP and Ketamine (=PCP-like compound) on the postsynaptic neuron impairs synaptic transmission including (1) long-term potentiation, notably in the hippocampus which alters memory (Neill et al., 2010; Ingram et al., 2018; Stringer and Guyenet, 1983), and (2) mismatch negativity which alters automatic auditory-change detection (Garrido et al., 2009).

Scopolamine acute model is of relevance for Schizophrenia. Administration of the muscarinic ACh receptor antagonist, scopolamine, produces attention, working memory and learning acquisition deficits in healthy volunteers that resemble to those exhibited by schizophrenic and demented patients (Tang, 2019; Gilles and Luthringer, 2007). The reduction of ACh release at the synapse by the presynaptic neuron impairs synaptic transmission including long-term potentiation, notably in the hippocampus which alters memory (More et al., 2016; Hirotsu et al., 1989).

### Other Applications:

In an aspect, the peptides or pharmaceutical compositions are used for preventing or treating a NMDAr and/or AMPAr deficiency related disease, notably anti-NMDAr encephalitis. In particular, the peptides are beneficial through an increase or enhancing of glutamatergic neurotransmission, as disclosed herein and supported by examples 4 and 5. The peptide increases, reestablishes or protects the glutamatergic neurotransmission.

In an aspect, the peptides or pharmaceutical compositions are used for treating vegetative state. In particular, the peptides are beneficial through an increase or enhancing of glutamatergic neurotransmission, as disclosed herein and supported by example 3. The peptide increases, reestablishes or protects the glutamatergic neurotransmission.

In an aspect, the peptides or pharmaceutical compositions are used for treating hypoxia-induced depression of synaptic transmission and/or hypoxic brain injury. In particular, the peptides are beneficial through a better and faster restoration of excitatory synaptic transmission, as disclosed herein and supported by examples 7 and 8. The peptide increases, reestablishes or protects the excitatory synaptic transmission.

In an aspect, the peptides or pharmaceutical compositions are used for treating bipolar disorder.

In an aspect, the peptides or pharmaceutical compositions are used for treating synaptic deficits resulting from a viral infection, especially in SARS CoV2 and in COVID-19 (particularly long-haulers sick persons).

In an aspect, the peptides or pharmaceutical compositions are used for treating or preventing a synaptopathy, more particularly the synaptic dysfunction in said synaptopathy. More precisely, the synaptopathy is one with impaired glutamatergic neurotransmission, especially linked to NMDAr and/or AMPAr, as disclosed herein.

In an aspect, the peptides or pharmaceutical compositions are used for treating or preventing a psychiatric disorder, such as autism, schizophrenia, bipolar dysfunction, and depression.

### CREB:

The transcription factor c-AMP-responsive element binding protein (CREB) is essential for activity-induced gene expression mediating memory formation (Silva et al., 1998). The CREB pathway responds to the increased calcium that results from neuronal activity.

Abnormalities of CREB expression is observed in the brain of individuals suffering from schizophrenia (Wang 2018). Evidence is provided by patients' postmortem pathological studies. The protein and gene levels of CREB and the binding activity of CREB to CRE in schizophrenic brains were significantly decreased in the cingulate gyrus (Yuan et al., 2010; Ren et al., 2014), an integral brain limbic system structure, which is involved with emotion, learning, and memory and found to be smaller and with lower neural activity in people with schizophrenia and bipolar disorder. Therefore, the CREB pathway may represent a promising target for the development of innovative interventions for schizophrenia and bipolar disorder.

The increase in phosphorylated CREB (pCREB) in T lymphocytes is significantly associated with clinical improvement in patients treated with antidepressants. In a study, the authors focused on patients treated only with psychotherapy to exclude direct pharmacological actions. After 6 weeks of psychotherapy, 17 patients responded to therapy; after 1-week pCREB increased significantly compared to the nonresponder group. (Koch 2009)

Serum levels of CREB are lower in Post-Traumatic Stress Disorder (PTSD) patients as compared to control psychiatrically healthy subjects. CREB levels did not change with trauma types in PTSD patients. CREB might be involved in the pathophysiology of PTSD (Olam 2019).

Impairment of CREB signaling has been well documented in addiction, Parkinsonism, schizophrenia, Huntington's disease, hypoxia, preconditioning effects, ischemia, alcoholism, anxiety, and depression (Sharma 2020).

The positive effect of the peptides of the invention on CREB is of interest in the present therapeutic indications, such as schizophrenia, hypoxia, bipolar disorder.

### Pharmaceutical Compositions:

The pharmaceutical compositions as used herein comprise as active ingredient a peptide or mixture of peptides as previously described, for example peptides of different amino acid composition or peptides of the same amino acid composition under oxidized and linear forms, and one or more pharmaceutically-acceptable vehicles, carriers or excipients.

The peptide compounds according to the invention may be used in a pharmaceutical composition, or in the manufacture of a medicament for preventing or treating basal excitatory synaptic transmission, notably glutamatergic neurotransmission, as disclosed herein.

In these compositions or medicaments, the active principle may be incorporated into compositions in various forms, i.e. in the form of solutions, generally aqueous solutions, or in freeze-dried form, or in the form of emulsion or any other pharmaceutically and physiologically acceptable form suited to the administration route.

Administration route may be a systemic route. Mention may be made in particular of the following injection or administration routes: intravenous, intrathecal, intraperitoneal, intranasal, subcutaneous, intramuscular, sublingual, oral, and combinations thereof.

Administration may also be local notably using intracerebral routes, especially intracerebroventricular administration.

The compositions containing one or more of the herein-disclosed peptides are sterile. These compositions are suitable for an administration leading to delivering the peptide(s) into the patient, e.g. in blood circulation. Delivery to the patient is delivery of a sufficient amount of the peptide(s), and this sufficient amount is correlated with the beneficial effect. The “pharmaceutical effect” may comprise increasing or enhancing glutamatergic transmission, as disclosed herein.

In some embodiments, the active principle in the pharmaceutical composition consists of (1) a linear peptide as disclosed herein, (2) an oxidized peptide as disclosed herein, (3) NX210, (4) NX218 or (5) a mixture of linear and oxidized peptides, such as in particular NX210 and NX218, in similar amounts or not, as disclosed above.

The active principle can be administered in a unit administration form, as a mixture with conventional pharmaceutical supports, carriers, excipients or vehicles, to animals and human beings. Suitable unit administration forms comprise oral-route forms such as tablets, gel capsules, powders, granules and oral suspensions or solutions, sublingual and buccal administration forms, aerosols; implants; subcutaneous, transdermal, topical, intraperitoneal, intramuscular, intravenous, subdermal, transdermal and intranasal administration forms and rectal administration forms.

Preferably, the pharmaceutical compositions contain carriers, excipients or vehicles which are pharmaceutically acceptable for a liquid formulation capable of being administered, e.g. injected to deliver the active principle in the patient, e.g. in blood stream. These may be in particular ready to use solutions, such as isotonic, sterile, saline solutions (monosodium or disodium phosphate, sodium, potassium, calcium or magnesium chloride and the like or mixtures of such salts), or dry, especially freeze-dried compositions which upon addition, depending on the case, of sterilized water or physiological saline, permit the constitution of administrable solutions.

The pharmaceutical forms include sterile aqueous solutions or dispersions; formulations including sesame oil, peanut oil or aqueous propylene glycol; and sterile powders for the extemporaneous preparation of sterile injectable solutions or dispersions. In all cases, the form must be sterile and must be fluid to the extent that easy syringability exists. It must be stable under the conditions of manufacture and storage and must be preserved against the contaminating action of microorganisms, such as bacteria and fungi.

Sterile injectable solutions are prepared by incorporating the active polypeptides in the required amount in the appropriate solvent followed by filtered sterilization.

Upon formulation, solutions will be administered in a manner compatible with the dosage formulation and in such amount as is therapeutically effective. The formulations are easily administered in a variety of dosage forms, such as the type of injectable solutions described above, but drug release capsules and the like can also be employed.

For suitable administration in an aqueous solution, for example, the solution should be suitably buffered if necessary and the liquid diluent first rendered isotonic with sufficient saline or glucose. These particular aqueous solutions are especially suitable for administration, e.g. intravenous, intramuscular, subcutaneous and intraperitoneal administration. In this connection, sterile aqueous media which can be employed will be known to those of skill in the art in light of the present disclosure. In addition to the compounds of the invention formulated for injection administration, such as intravenous or intramuscular injection, other pharmaceutically acceptable forms include, e.g. tablets or other solids for oral administration; liposomal formulations; time release capsules; and any other form currently used, and delivering the active principle.

One dose of peptide(s) is expressed in weight of peptide per patient body weight (kg) and may range from about 1 μg/kg to about 1 g/kg, in particular from about 10 μg/kg to about 100 mg/kg, e.g. from about 50 μg/kg to about 50 mg/kg.

The dosage regimen may comprise a single administration or repeated administrations. According to an embodiment, repeated administrations may comprise administering one dose per day of treatment, for example one dose every day or every 2 or 3 days over a treatment period. According to another embodiment, repeated administrations may comprise administering at least two doses per day of treatment, for example 2, 3 or more doses per day over a treatment period. In these embodiments, a treatment period may be 1, 2, 3, 4, 5, 6, 7, 8, 9, 10, 15, 20, 25, 30, 35, 40, 45 or more days (e.g. up to 6 months). The treatment is designed so that the patient keeps “benefit” from this treatment over a “period of time”. “Benefit” may comprise the “pharmaceutical effect” mentioned above, say the peptides are beneficial to the subject suffering or in risk of suffering of one of these diseases, through an increase, enhancing or restoration of glutamatergic neurotransmission. The peptides thus allow for an improvement (increase, reestablishing or protecting) of the glutamatergic neurotransmission and thus an improvement of the functional or intellectual faculties or cognition involving the synapses for which the peptide increases, reestablishes or protect the glutamatergic neurotransmission. This “period of time” may depend from the dosage regimen and from the patient itself, e.g. the severity of the illness and the responsiveness of the patient to the regimen dose. “Improvement” comprises “partial improvement” and “total improvement”. It is said “partial” when in the subject it is observed a partial recovery of the glutamatergic neurotransmission and of the physiological functions; with respect to the initial status of the subject before treatment, there is a significant improvement of these, however it remains significantly below with respect to healthy subjects. “Total recovery” means that the subject recovered glutamatergic neurotransmission and physiological functions; or that they are not significantly different than healthy subjects.

### Methods of Treatment:

In an aspect, the invention relates to a method of treating a subject in need thereof in order to enhance basal excitatory synaptic transmission, notably glutamatergic neurotransmission, the method comprising administering to the subject a therapeutic amount of a SCO-Spondin derived peptide and a pharmaceutically acceptable vehicle or excipient.

The subject in need thereof may be a subject having a reduced basal excitatory synaptic transmission, notably a reduced glutamatergic neurotransmission (in particular NMDAr related glutamatergic neurotransmission, especially linked to GluN2A subunit). In particular, this reduced transmission or neurotransmission is characterized by a postsynaptic current below the normal. As explained before, the enhanced transmission or neurotransmission may be characterized by an increased post-synaptic current amplitude, at least an increased NMDAr post-synaptic current amplitude.

The subject in need thereof may be a subject having a normal basal excitatory synaptic transmission, notably glutamatergic neurotransmission. The enhanced transmission or neurotransmission may be characterized by an increased post-synaptic current amplitude, at least an increased NMDAr post-synaptic current amplitude, above the basal value.

Preferably, the SCO-spondin derived peptide is selected from the group consisting of the peptides of sequence SEQ ID NO: 1 or 2. More particularly, the peptide is selected from the group consisting of the peptides of sequence SEQ ID NO: 3-63. Preferably, the peptide is NX218.

In an aspect of the invention, the method treats a disease or condition selected from the group consisting of psychiatric disorders, drug addiction, viral, especially coronavirus infections and their related neurological symptoms, NMDAr and/or AMPAr deficiency related disease, vegetative state and hypoxic brain injury. In particular, the peptides are beneficial through an increase, enhancing or restoration of glutamatergic neurotransmission, as disclosed herein.

In some aspects, the method treats a Schizophrenia, a Drug addiction (e.g. PCP, Ketamine and Scopolamine), NMDAr and/or AMPAr deficiency related disease, a Vegetative state, as disclosed herein.

In some aspect, the method prevents and/or treats hypoxia-induced depression of synaptic transmission or hypoxic brain injury, as disclosed herein.

In an aspect, the invention relates to a method of treating a subject in need thereof in order to increase, enhance or restore synaptic transmission, especially in the hippocampus, the method comprising administering to the subject a therapeutic amount of a SCO-Spondin derived peptide and a pharmaceutically acceptable vehicle or excipient.

In an aspect, the invention relates to a method of treating a subject in need thereof in order to prevent or treat the effect of hypoxia on excitatory postsynaptic potentials, especially in the hippocampus, the method comprising administering to the subject a therapeutic amount of a SCO-Spondin derived peptide and a pharmaceutically acceptable vehicle or excipient.

The peptide may in particular allow keeping normal synaptic transmission or close to normal, and/or recovering normal synaptic transmission or increasing synaptic transmission.

The peptide may in particular allow keeping normal EPSPs or close to normal, and/or recovering normal EPSPs or increasing EPSPs.

The other characteristics disclosed in this specification applies to these methods of treatment. In particular, the described “for use” or “use for” are to be regarded as basis for a “method of use”.

### Further Definitions

Administration or use of “peptide” or “peptides” or “peptide(s)”, is a generic wording, and the invention encompasses administration or use of one single peptide or more than one single peptide, i.e. the administration or use of at least two peptides according to the present disclosure. Thus, in the present disclosure the singular or the plural is not limited unless indicated to the contrary, and may each time encompass one single peptide, or at least two peptides. The same apply to the equivalent wording “peptide compound” that may be used interchangeably for “peptide”.

“Treating”, “treated”, or “treat”, means delivering an amount of peptide compound according to the invention to a subject. These terms as used herein refers to a therapeutic wherein the object is to slow down (lessen) an undesired physiological condition, disorder or disease, or to obtain beneficial or desired clinical results. For the purposes of this invention, beneficial or desired clinical results include, but are not limited to, modulating, stabilizing, preferably increasing or enhancing, glutamatergic neurotransmission; diminishment of the symptoms resulting from impaired glutamatergic neurotransmission; stabilization (i.e. not worsening) of the state of the symptoms, condition, disorder or disease; delay in onset or slowing of the progression of the symptoms, condition, disorder or disease; amelioration of the symptoms, condition, disorder or disease state; and remission (whether partial or total), with substantial reestablishment of glutamatergic neurotransmission, or enhancement or improvement of the condition, disorder or disease. The terms “treating”, “treated” or “treat” may include preventing, suppressing, repressing, ameliorating, or completely eliminating the disease symptoms linked to glutamatergic neurotransmission. Preventing the disease may involve administering a composition of the present invention to a subject prior to onset of the disease symptoms linked to glutamatergic neurotransmission. Suppressing the disease symptoms linked to glutamatergic neurotransmission may involve administering a composition of the present invention to a subject after induction of the disease but before its clinical appearance. Repressing or ameliorating the disease symptoms linked to glutamatergic neurotransmission may involve administering a composition of the present invention to a subject after clinical appearance of these disease symptoms.

“Effective amount”, “sufficient amount”, and similar such as “therapeutically effective amount”, are used interchangeably herein unless otherwise defined, and means a dosage of a peptide or peptides of the invention effective for periods of time necessary to achieve the desired therapeutic result. An effective dosage may be determined by a person skilled in the art and may vary according to factors such as the disease state, age, sex, and weight of the individual, and the ability of the drug to elicit a desired response in the individual. This term as used herein may also refer to an amount effective at bringing about a desired in vivo effect in a subject, in particular a stabilization or, preferably, an increase of glutamatergic neurotransmission. A therapeutically effective amount may be administered in one or more administrations (e.g., the composition may be given as a preventative treatment or therapeutically at any stage of disease progression, before or after symptoms, and the like), applications, or dosages, and is not intended to be limited to a particular formulation, combination, or administration route. It is within the scope of the present disclosure that the peptide(s) may be administered at various times during the course of treatment of the subject. The times of administration and dosages used will depend on several factors, such as the goal of treatment (e.g., treating vs. preventing), condition of the subject, etc., and can be readily determined by one skilled in the art. A therapeutically effective amount is also one in which any toxic or detrimental effects of substance are outweighed by the therapeutically beneficial effects. A “prophylactically effective amount” refers to an amount effective, at dosages and for periods of time necessary, to achieve the desired prophylactic result. Typically, since a prophylactic dose is used in subjects prior to or at an earlier stage of disease, the prophylactically effective amount may be less than the therapeutically effective amount. “Effective amount”, “sufficient amount”, may also take into account the combination of different peptides if one considers the amount of the peptides separately, and/or the combination with another active principle, owing to which, for example, the dose of one or the two drugs in the combination may be lowered by result of a combined effect or a synergic effect.

The terms “comprise(s),” “include(s),” “having,” “has,” “can,” “contain(s),” and variants thereof, as used herein, are intended to be open-ended transitional phrases, terms, or words that do not preclude the possibility of additional acts or products, such as peptides, compounds or drugs. The singular forms “a,” “and” and “the” include plural references unless the context clearly dictates otherwise. The present disclosure also contemplates other embodiments “comprising,” “consisting of” and “consisting essentially of,” the embodiments or elements presented herein, whether explicitly set forth or not.

“Patient or subject” means an animal, especially a mammal, including a human. In an embodiment, the subject is a human. In other embodiments, the subject is a big or a farm animal, a companion animal (e.g. cat, dog) or a sport animal (e.g. horse).

In an embodiment of the present use or method of treatment, may be excluded one or several of the following diseases: Parkinson's Disease (PD), Multiple Sclerosis (MS), Myopathies, non-brain nervous system injury, such as Spinal Cord Injury (SCI) or Optic Nerve Injury (ONI), tauopathies (i.e. Tau positives neurodegenerative diseases, including any of Alzheimer's Disease (AD), Progressive Supranuclear Palsy (PSP), Tau positive Fronto-Temporal Dementia such as Pick's disease, dementia with Lewy bodies, corticobasal degeneration, Niemann-Pick type C disease, chronic traumatic encephalopathy including dementia pugilistica, postencephalitic parkinsonism); may be excluded individually Alzheimer's Disease (AD); Progressive Supranuclear Palsy (PSP); Tau positive Fronto-Temporal Dementia such as Pick's disease; dementia with Lewy bodies; corticobasal degeneration; Niemann-Pick type C disease; chronic traumatic encephalopathy including dementia pugilistica; postencephalitic parkinsonism; cerebral ischemia; CNS neuronal traumas (i.e spinal cord or brain injuries) pathologies; viral neurodegenerations; Amyotrophic Lateral Sclerosis; Spinal Muscular Atrophy; Huntington's Disease; prion disease; PSP; multiple system atrophy; adrenoleukodystrophy; Down syndrome.

The present invention will now be described in more details using non-limiting examples referring to the figures:

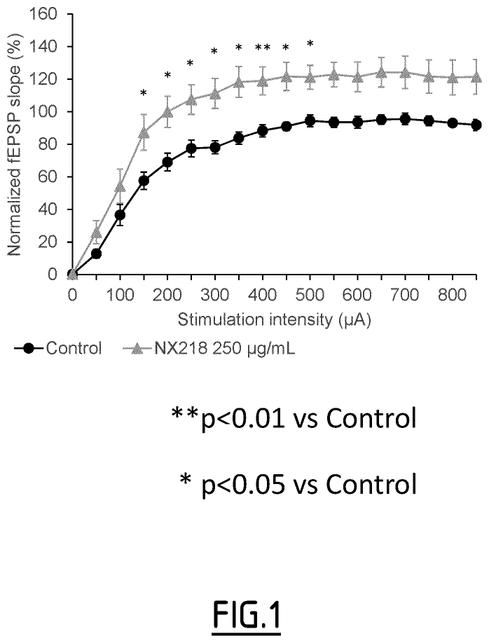

FIG. 1: Normalized average slope of fEPSP evoked during input-output (I/O) responses in somatosensory cortex after stimulation of ventrobasal thalamic nucleus in the presence of vehicle and NX218 peptide. The data are represented as means±SEM and analysed using a repeated two-way ANOVA followed by Šidák post hoc comparison test. Using 2-way ANOVA, a statistical increase of fEPSP slope was observed after NX218 peptide application compared with vehicle alone (p=0.0027 between treatment groups), specifically for stimulation intensities of 150 (*, p=0.049), 200 (*, p=0.034), 250 (*, p=0.044), 300 (*, p=0.016), 350 (**, p=0.009), 400 (*, p=0.037) and 450 (*, p=0.036).

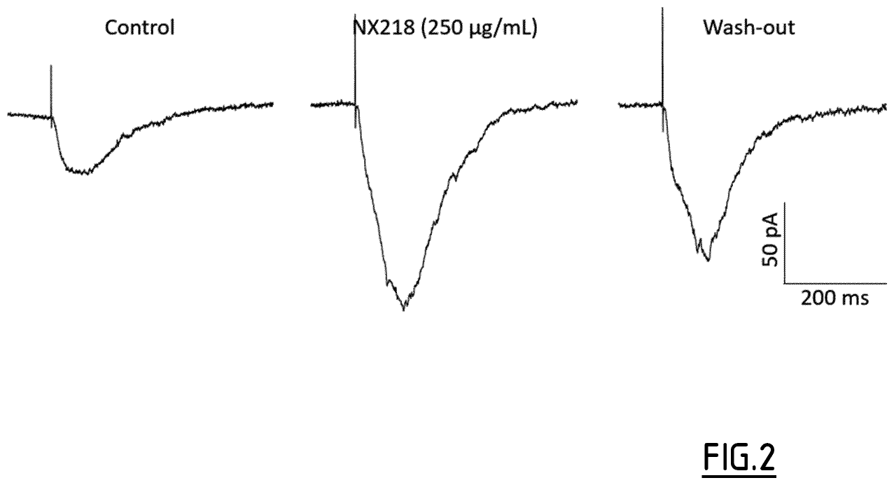

FIG. 2: Examples of evoked isolated NMDA excitatory postsynaptic currents in the absence and in the presence of NX218.

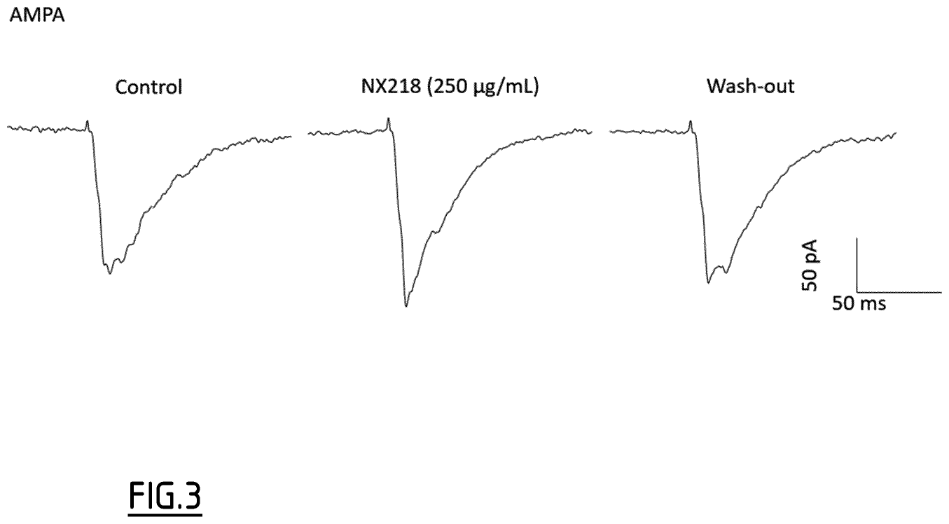

FIG. 3: Examples of evoked isolated AMPA excitatory postsynaptic currents in the absence and in the presence of NX218.

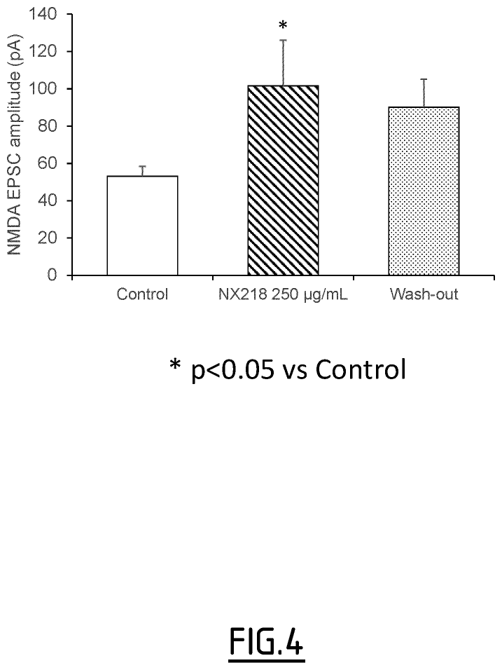

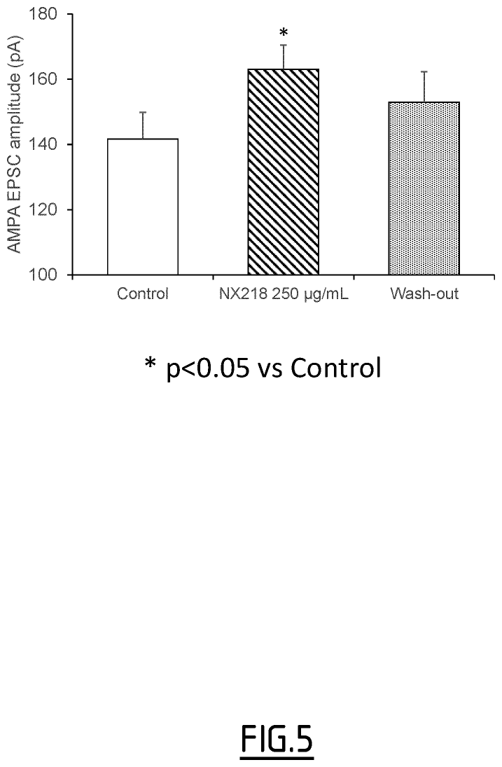

FIG. 4: Amplitudes of evoked isolated NMDA excitatory postsynaptic currents in the absence and in the presence of NX218.

FIG. 5: Amplitudes of evoked isolated AMPA excitatory postsynaptic currents in the absence and in the presence of NX218.

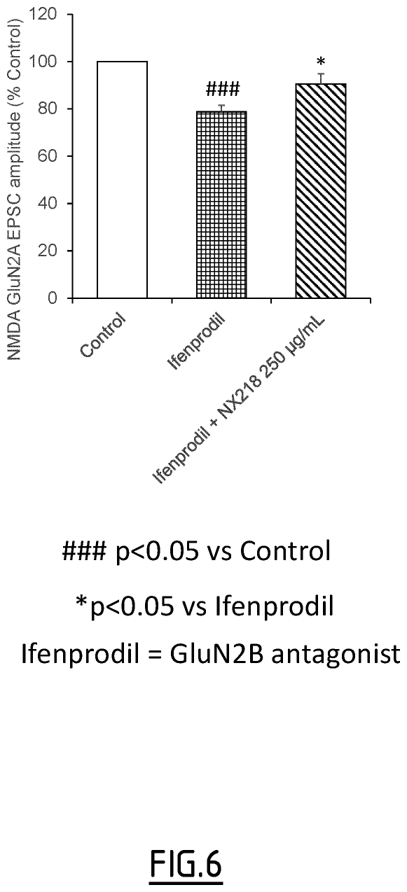

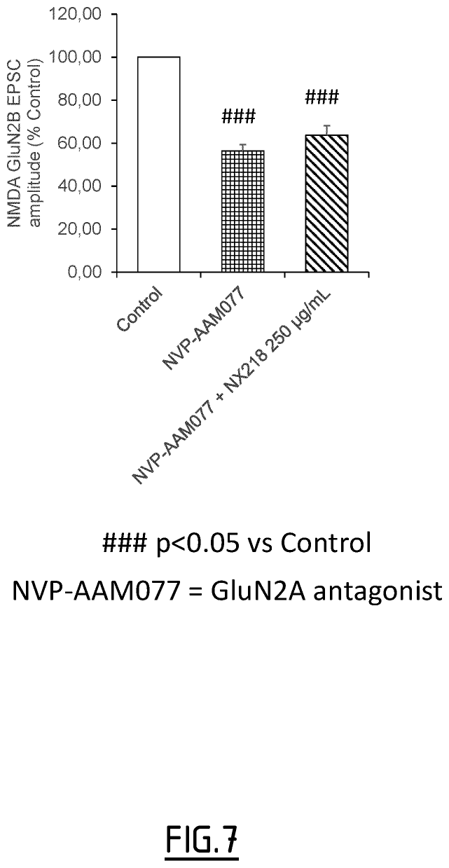

FIG. 6: Normalized amplitude of evoked isolated NMDA excitatory postsynaptic currents before (baseline) and after sequential addition of Ifenprodil (3 μM) (GluN2B antagonist) and NX218 peptide (250 μg/mL). NMDA EPSCs amplitude is significantly decreased by the application of Ifenprodil (3 μM) (Absolute: ****, p<0.0001; Normalized: ****, p<0.0001, RM one-way ANOVA). Addition of NX218 (250 μg/mL) significantly increased NMDA excitatory postsynaptic currents (Baseline vs Ifendrodil+NX218: Absolute ns; Normalized ns; Ifenprodil vs Ifendrodil+NX218: Absolute: #, p=0.0529; Normalized: *, p=0.0263, RM one-way ANOVA).

FIG. 7: Normalized amplitude of evoked isolated NMDA excitatory postsynaptic currents before (baseline) and after sequential addition of NVP-AAM077 (0.4 μM) (GluN2B antagonist) and NX218 peptide (250 μg/mL). NMDA EPSCs amplitude is significantly decreased by the application of NVP-AAM077 (0.4 μM) (Absolute: ****, p<0.0001; Normalized: ****, p<0.0001, RM one-way ANOVA). Addition of NX218 (250 μg/mL) has not significant effect on EPSC amplitude (Baseline vs NVP-AAM077+NX218: Absolute: ***, p=0.0002; Normalized: ****, p<0.0001; NVP-AAM077 vs NVP-AAM077+NX218: Absolute: ns; Normalized: ns, RM one-way ANOVA).

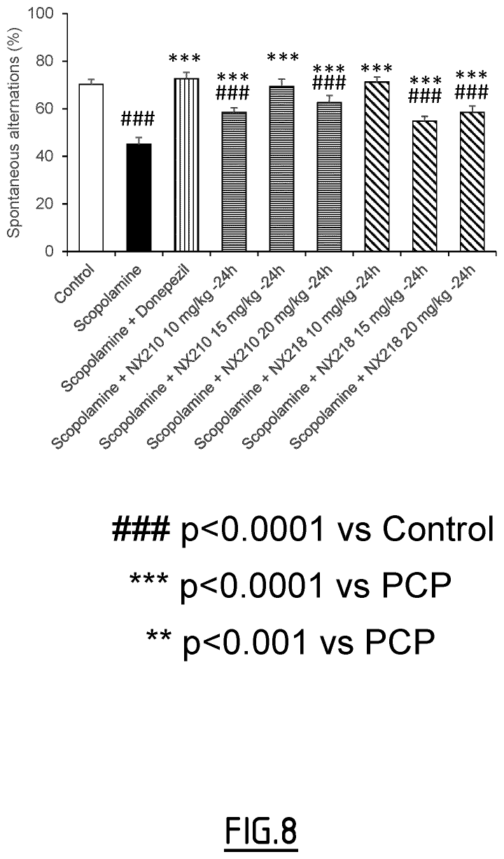

FIG. 8: Effect of intraperitoneal administration of NX210 or NX218 on scopolamine-induced spatial short-term working memory deficits in mice: Y-maze locomotion. NX 210 or NX218 were administered at 3 doses (10, 15 and 20 mg/kg) 24 h before the test. Donepezil (DPZ) was administered 1 h before the test (positive control). Doses are expressed in mg per kg with n is 6 per group; *** p<0.001 vs. the Veh/Veh group, ###p<0.001 vs. the Veh/Scop group, One-Way ANOVA followed by Dunnett's test.

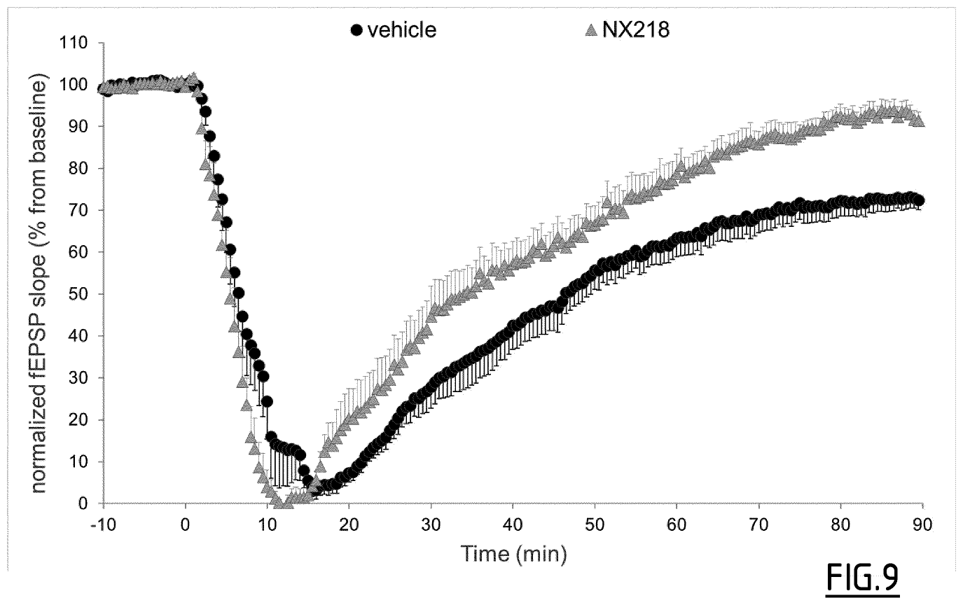

FIG. 9: Time course of the averaged normalized f-EPSP slope evoked by Schaffer collateral stimulation during and after oxygen/glucose deprivation (OGD) in mouse hippocampal slices superfused with vehicle (n=9) or NX218 at 250 μg/mL (n=9). Black circles represent the response in vehicle-treated slices (control slices, n=9) before, during and after OGD induction. Gray triangles represent the response in NX218-treated slices (treated slices, n=9) before, during and after OGD induction.

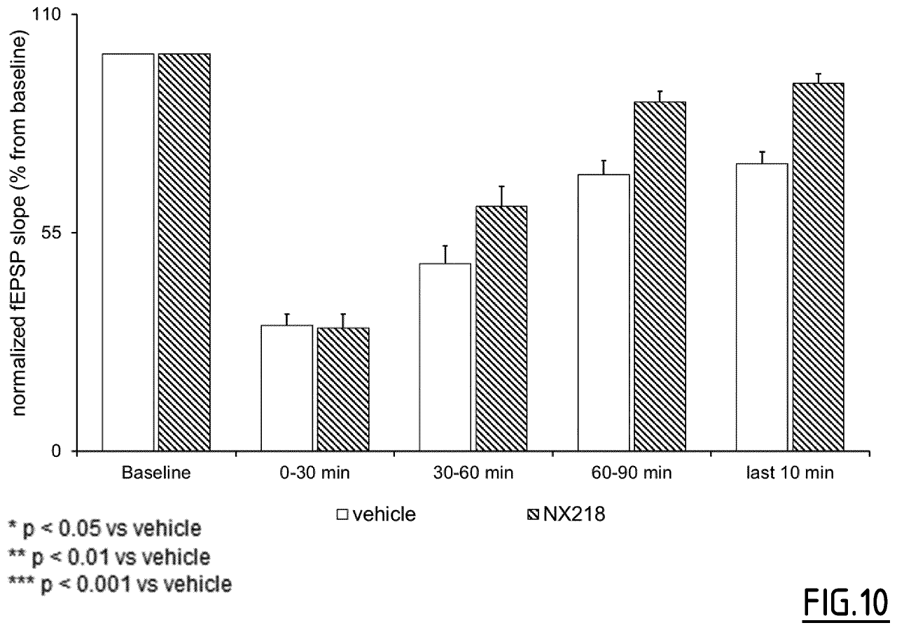

FIG. 10: Bar graphs of the averaged normalized f-EPSP slope evoked by Schaffer collateral stimulation during and after oxygen/glucose deprivation (OGD) in mouse hippocampal slices superfused with vehicle (n=9) or NX218 at 250 μg/mL (n=9). White bars represent f-EPSP slopes in vehicle-treated slices (control slices, n=9) before (baseline) and different timepoints after the start of OGD induction. Hatched bars represent f-EPSP slopes in NX218-treated slices (treated slices, n=9) before (baseline) and different timepoints after the start of OGD induction. P-value summaries: * p<0.05, ** p<0.01, *** p<0.001.

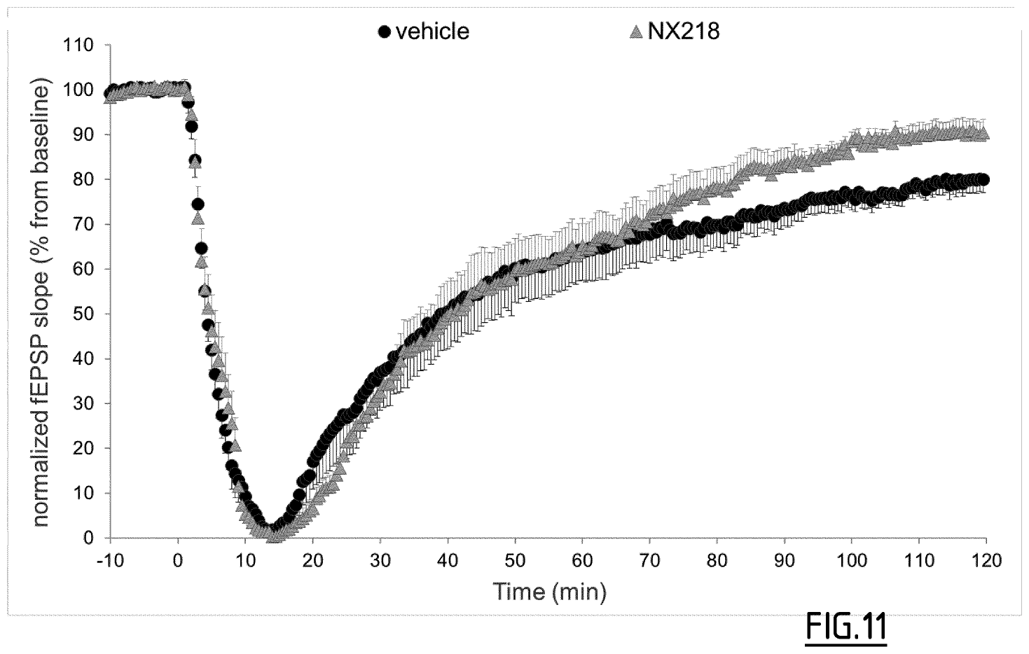

FIG. 11: Time course of the averaged normalized f-EPSP slope evoked by Schaffer collateral stimulation during and after oxygen/glucose deprivation (OGD) in mouse hippocampal slices superfused with vehicle (n=10) or NX218 at 250 μg/mL (n=10) from the end of OGD. Black circles represent the response in vehicle-treated slices (control slices, n=10) before, during and after OGD induction. Gray triangles represent the response in NX218-treated slices (treated slices, n=10) before, during and after OGD induction.

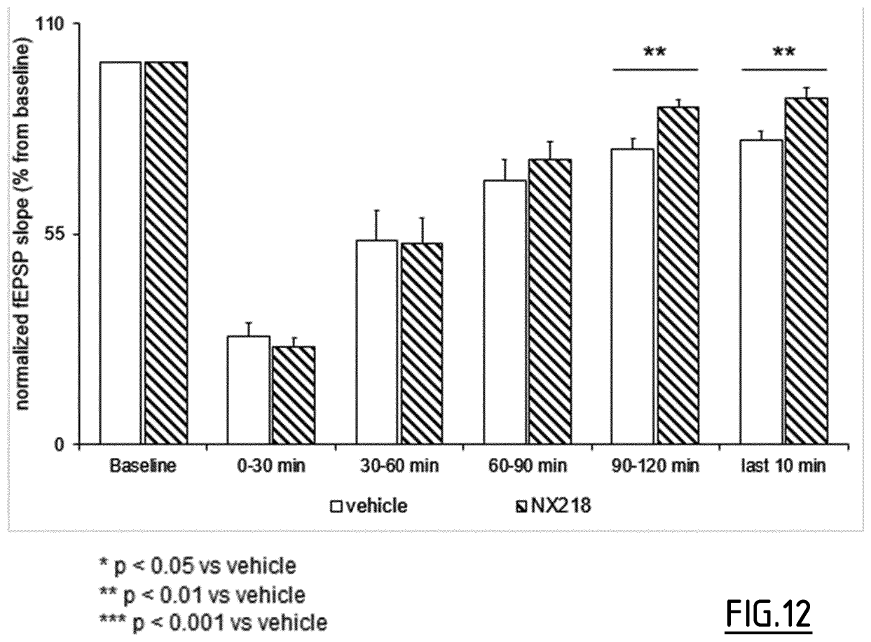

FIG. 12: Bar graph of the averaged normalized f-EPSP slope evoked by Schaffer collateral stimulation during and after oxygen/glucose deprivation (OGD) in mouse hippocampal slices superfused with vehicle (n=10) or NX218 at 250 μg/mL (n=10) at the end of OGD. White bars represent f-EPSP slopes in vehicle-treated slices (control slices, n=10) before (baseline) and different timepoints after the start of OGD induction. Hatched bars represent f-EPSP slopes in NX218-treated slices (treated slices, n=10) before (baseline) and different timepoints after the start of OGD induction. P-value summaries: * p<0.05, ** p<0.01, *** p<0.001.

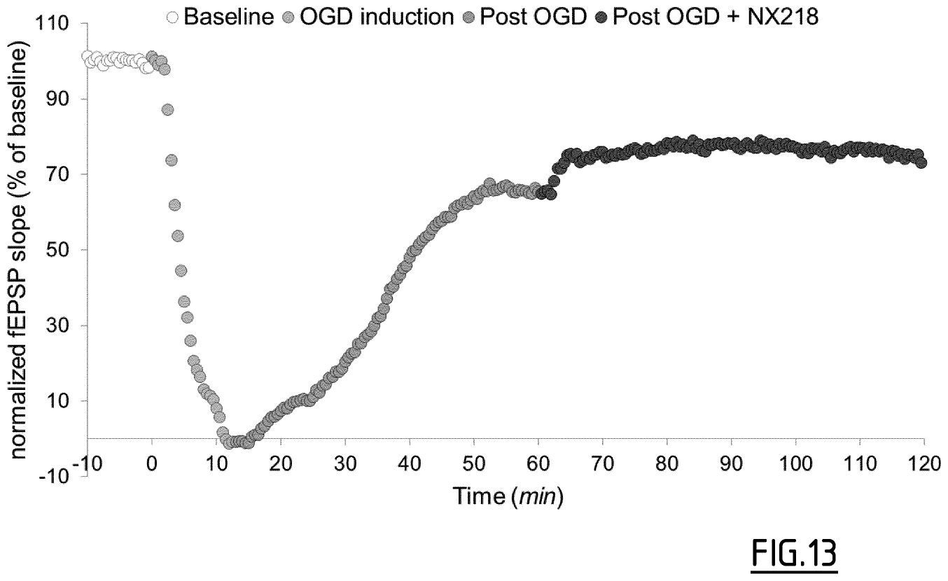

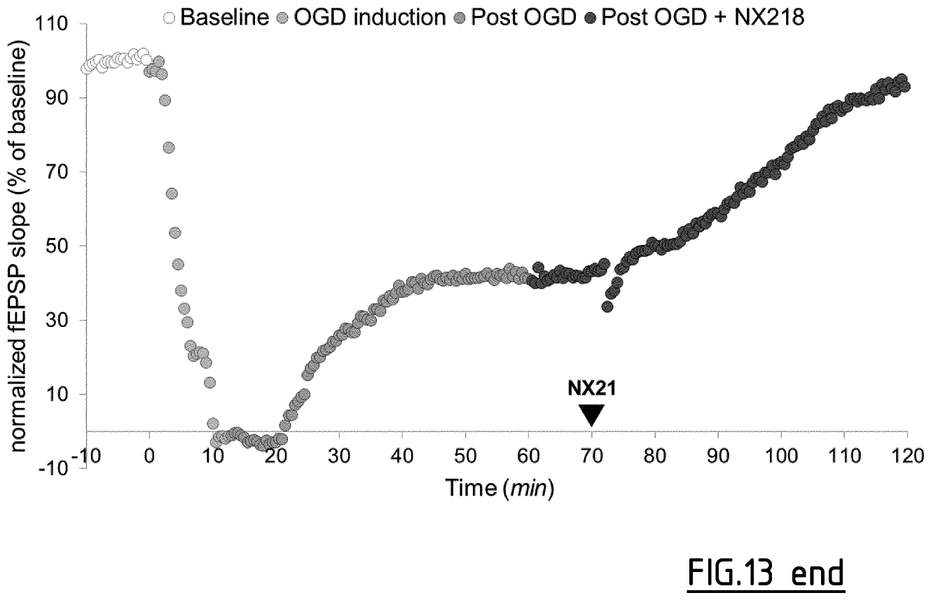

FIG. 13: Time course of the averaged normalized f-EPSP slope evoked by Schaffer collateral stimulation during and after oxygen/glucose deprivation (OGD) in mouse hippocampal slices superfused with NX218 at 250 μg/mL during recovery plateau, at T=60 min and T=70 min after OGD initiation (n=1 per timepoint).

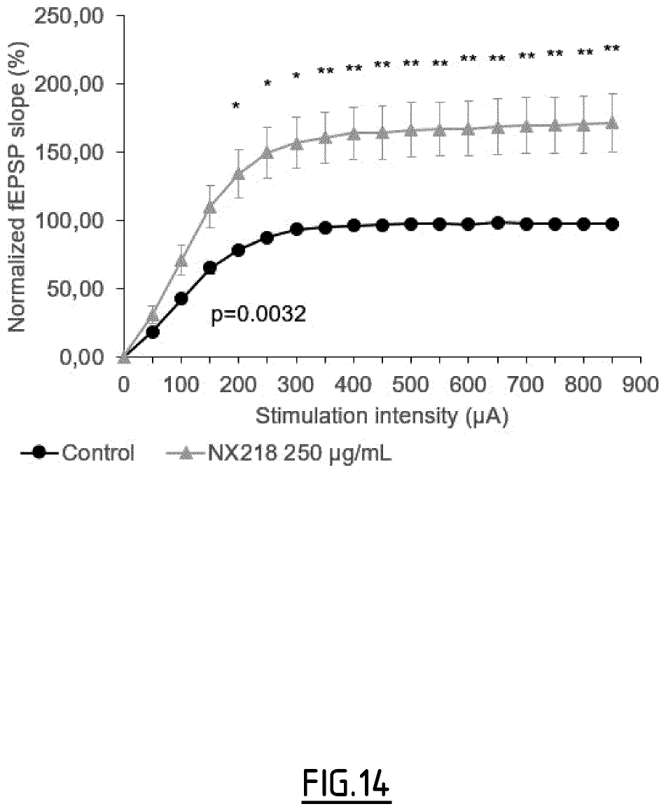

FIG. 14: Normalized slopes of fEPSPs evoked during I/O recordings in the CA1 area following Schaffer collateral stimulation in hippocampal slices sequentially superfused with vehicle and NX218 at 250 μg/mL. Input-output (I/O) curves were obtained by plotting the fEPSPs slope against different intensities of presynaptic stimulation (from 0 to 850 μA with a step of 50 μA). All data was normalized from the fEPSP maximum slope recorded in control condition (vehicle perfusion). Data are represented as means±SEM and analysed using a two-way ANOVA followed by Sidak's multiple comparison test. N=10 slices/group. **p<0.01 vs Control. * p<0.05 vs Control.

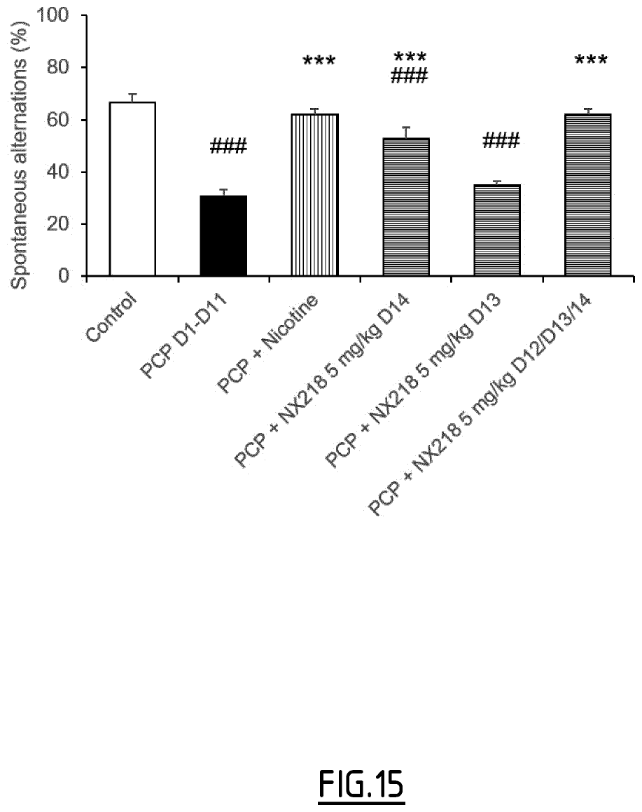

FIG. 15: Spatial memory was assessed as T-maze spontaneous alternation in vehicle and subchronic PCP mice treated with vehicle, Nicotine 0.4 mg/kg 30 min, NX218 5 mg/kg 2 h (D14), NX218 5 mg/kg 24 h (D13), NX218 5 mg/kg 48 h+24 h+2 h (D12, D13 and D14, respectively). Data are presented as mean±s.e.m. of n=10 for all groups, except for the group NX218 5 mg/kg 24 h (n=9) where significant outlier was identified via QuickCalcs. One way-ANOVA followed by Fisher's Protected Least Significant Difference for pairwise comparison: ***p<0.001 vs subchronic PCP. ###p<0.0001 vs Control. ***p<0.0001 vs PCP.

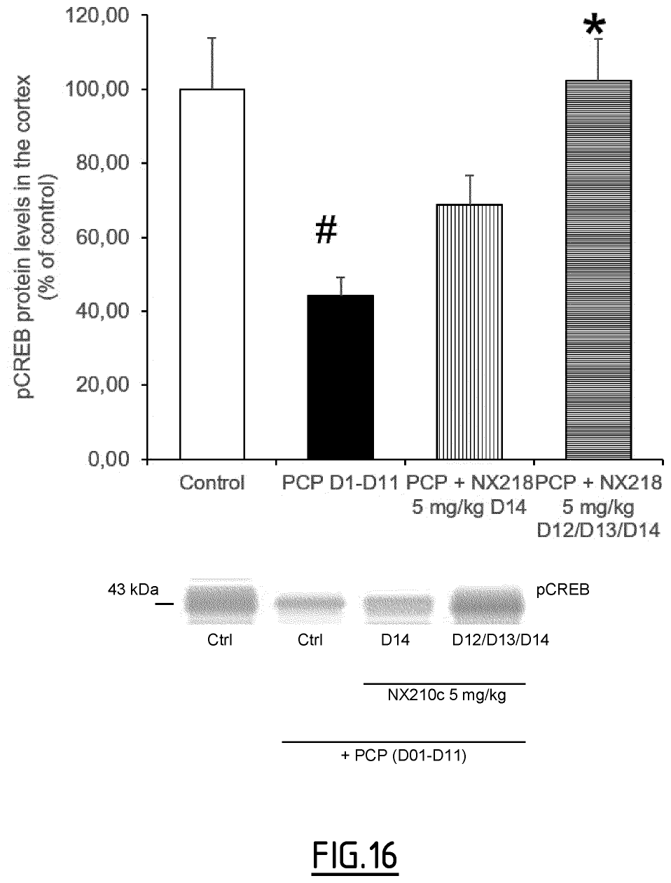

FIG. 16: Protein level of pCREB in cortical samples in a mouse model of cognitive deficits induced by the subchronic administrations of PCP. Results are expressed as a percentage of control condition as mean+/−SEM (n=4-5). One-way ANOVA followed by Tukey's test (*, p<0.05 was considered significant from PCP group). #p<0.05 vs Control. * p<0.05 vs PCP.

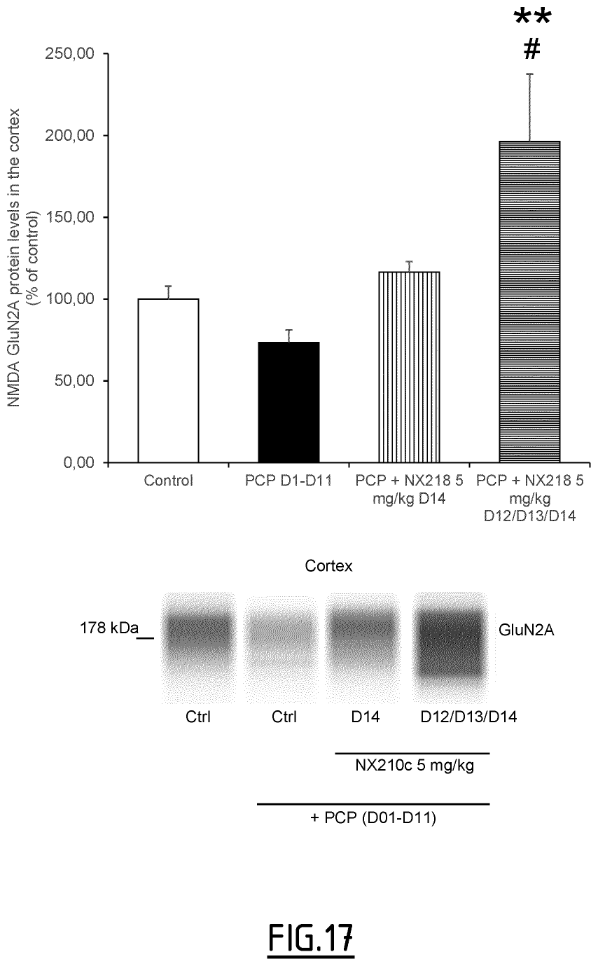

FIG. 17: Level of GluN2A in cortical samples in a mouse model of cognitive deficits induced by the subchronic administrations of PCP. Results are expressed as a percentage of control condition as mean+/−SEM (n=4-5). One-way ANOVA followed by Tukey's test (*, p<0.05 was considered significant from PCP group). #p<0.05 vs Control. **p<0.01 vs PCP.

### EXAMPLE 1: SYNTHESIS OF NX PEPTIDES

The manufacturing process of the peptides of sequence SEQ ID NO: 1, 2, or of any of the sequences 3-63, and especially those used in the Part Example, such as NX210 (SEQ ID NO: 3), is based on Solid-Phase Peptide Synthesis applying N-α-Fmoc (side chain) protected amino acids as building blocks in the assembly of the peptide. The protocol employed consists of a coupling of the C-terminal Glycine N-α-Fmoc-protected amino acid bound to an MPPA linker on the MBHA resin, followed by Fmoc coupling/deprotection sequences. After assembly of the peptide on the resin, a step of simultaneous cleavage of the peptide from the resin and deprotection of the side chains of amino acid is carried-out.

The crude peptide is precipitated, filtered and dried. Prior to purification by preparative reverse phase chromatography, the peptide is dissolved in an aqueous solution containing acetonitrile. The purified peptide in solution is the concentrated before undergoing an ion exchange step to obtain the peptide in the form of its acetate salt.

The skilled person may refer for further detail of synthesis to U.S. Pat. No. 6,995,140 and WO2018146283, and for the oxidized forms of the peptides disclosed herein, to WO 2017/051135, all incorporated herein by reference.

The skilled person further has access to the standard methods to produce any of the disclosed peptides of the invention including the N-ter and C-ter modified or protected peptides. Concerning the acetylation and/or the amidation of the peptides at the N-terminal and C-terminal respectively, the skilled person may refer to standard techniques, e.g. those described in Biophysical Journal Volume 95 November 2008 4879-4889, also incorporated by reference.

### EXAMPLE 2: SYNTHESIS OF CYCLIC NX PEPTIDES

The polypeptide of sequence W-S-G-W-S-S-C-S-R-S-C-G was added to Human Serum Albumin (HSA) in a 1:1 ratio and incubated for 1 to 3 hours with stirring in air at room temperature. By using HPLC, we observed the formation of a peak corresponding to the polypeptide sequence W-S-G-W-S-S-C-S-R-S-C-G in which the 2 cysteines are linked by a disulfide bridge. After removal of the albumin by precipitation, the product was then purified and analyzed by HPLC. The use of a different ratio of albumin and of polypeptide corresponding to the sequence W-S-G-W-S-S-C-S-R-S-C-G makes it possible to influence the cyclization rate and the final yield of cyclisation, while knowing that a smaller amount of albumin is easier to eliminate. Cyclized compound is NX218.

The skilled person may refer for further detail of synthesis to WO2017051135. This document is incorporated herein by reference.

### EXAMPLE 3: NX218 EFFECT ON SYNAPTIC TRANSMISSION MEASURED ON MICE BRAIN SLICES IN THE THALAMOCORTICAL REGION

To determine the effect of NX218 peptide on thalamocortical synaptic response. Cortical field excitatory postsynaptic potentials (fEPSPs) were recorded in response to ventrobasal thalamic stimulations during focal application of NX218 peptide on the thalamic radiations on mice brain slices. Comparison of postsynaptic responses in control versus NX218 peptide conditions were assessed.

### Mice Brain Slices Preparation:

Five (C57Bl6/J) male mice 4-5 weeks-old were obtained from Charles River, France and housed in an animal facility. Animal care was compliant with national and local Ethics committee recommendations. Mice were deeply anesthetized by inhalation of isoflurane then decapitated. The dissected brain was quickly placed in ice-cold oxygenated (95% O₂/5% CO₂) solution containing 214 mM sucrose, 2.5 mM KCl, 1.25 mM NaH₂PO₄, 26 mM NaHCO₃, 2 mM MgSO₄, 2 mM CaCl₂ and 10 mM D-glucose.

10 Thalamocortical slices (2 slices per mouse) were prepared as described in Agmon and Connors (1991) and Varela et al. (2013). After its recovery, the brain was placed on a support allowing to elevate the caudal part of the brain until dorsal surface forms a 10-degree angle with the horizontal plane. Then, a section at 55 degrees with respect to the midline was performed and the rostral part was removed. In the slicing chamber, the brain was glued on the sectioned face. Then, the brain was sliced with a thickness of 400 μm. Slices were immediately transferred to a holding chamber filled with an artificial cerebrospinal fluid (aCSF) composed by 124 mM NaCl, 2.5 mM KCl, 1.25 mM NaH₂PO₄, 26 mM NaHCO₃, 2 mM MgSO₄, 2 mM CaCl₂ and 10 mM D-glucose. The holding chamber was continuously oxygenated and maintained at 35° C. After a recovery period of 30 min, slices were incubated at room temperature for a minimum of 30 min.

### Electrophysiological Recordings:

For electrophysiological recordings, a single slice was placed in the recording chamber (room temperature) submerged and continuously superfused at a constant rate (2 mL/min) with gassed (95% O₂, 5% CO₂) aCSF for the reminder of the experiment.

A bipolar tungsten stimulating electrode was placed in the ventrobasal nucleus of the thalamus (the ventrobasal nucleus of the thalamus contains mostly glutamatergic neurons and few GABAergic interneurons) and extracellular field potential was recorded in the somatosensory cortex using a glass microelectrode.

Synaptic transmission Input/Output (I/O) curves were constructed to assess changes in the synaptic transmission, using a range of stimulus intensities from 0 μA to 850 μA with 50 μA intervals. Increasing stimulation intensities results in a linear increase until a maximal plateau in vehicle conditions.

Signals were amplified with an Axopatch 200B amplifier (Molecular Devices, Union City, CA) digitized by a Digidata 1322A interface (Axon Instruments, Molecular Devices, US) and sampled at 10 kHz. Recordings were acquired using Clampex (Molecular Devices) and analyzed with Clampfit (Molecular Devices). Experimenters were blinded to treatment groups for all experiments.

### NX218 Drug Delivery System:

NX218 or vehicle was delivered in the thalamic radiations region by fast focal perfusion. The system consists of syringes containing the respective solutions with tubing lines fusing to a low dead-volume manifold mounted to a micromanipulator. The tip (300-μm diameter) mounted to the manifold was located within a desired distance of less than 1 mm of the target region, with a flow rates of approximately 0.2 mL/min. Tubing lines were controlled by electrical pinch valves which were opened and closed by transistor-transistor logic signals sent out from the DigiData system and parameters set in the perfusion and recording protocol in Clampex 10.3.

### Results

Data were analyzed by measuring the slope of individual fEPSPs by linear fitting using Clampfit. The slope of fEPSPs was plotted against different intensities of stimulation (from 0 to 850 μA). NX218 effect was assessed by changes in fEPSP slope, expressed as the percentage of the maximal value of the I/O curve during vehicle perfusion. The results from 10 validated slices are presented in Table 1 and FIG. 1.

TABLE 1
Normalized average slope of fEPSP evoked during input-output (I/O) responses
in somatosensory cortex after stimulation of ventrobasal thalamic nucleus in the
presence of vehicle and NX218 peptide

| | | Stimulation intensity(pA) | | | | | | | | |
|---|---|---|---|---|---|---|---|---|---|---|
| | | 0 | 50 | 100 | 150 | 200 | 250 | 300 | 350 | 400 |
| Normalized | Mean Control | 0.24 | 12.83 | 36.60 | 57.64 | 69.10 | 77.45 | 78.13 | 83.76 | 88.29 |
| fEPSP | Mean NX218 | 0.37 | 26.12 | 54.34 | 87.31 | 99.95 | 107.48 | 111.20 | 118.22 | 118.91 |
| slope | SEM Control | 0.72 | 2.75 | 6.42 | 5.29 | 5.42 | 5.21 | 3.99 | 3.78 | 3.74 |
| (%) | SEM NX218 | 0.38 | 7.08 | 10.47 | 10.95 | 9.54 | 9.09 | 9.17 | 9.47 | 8.63 |
| | | Stimulation intensity(pA) | | | | | | | | |
| | | 450 | 500 | 550 | 600 | 650 | 700 | 750 | 800 | 850 |
| Normalized | Mean Control | 90.97 | 94.44 | 93.48 | 93.57 | 95.11 | 95.52 | 94.41 | 93.00 | 91.85 |
| fEPSP | Mean NX218 | 121.63 | 121.16 | 122.81 | 121.42 | 124.24 | 124.14 | 121.65 | 120.97 | 121.41 |
| slope | SEM Control | 2.59 | 3.72 | 3.16 | 3.71 | 2.83 | 3.55 | 2.90 | 2.23 | 3.39 |
| (%) | SEM NX218 | 8.63 | 7.33 | 7.50 | 9.10 | 9.09 | 9.99 | 10.21 | 10.72 | 10.64 |

In this study, somatosensory cortical fEPSPs were recorded in response to ventrobasal thalamic stimulations during vehicle and NX218 peptide application in ex vivo cerebral slices from C57Bl6/J mice. A significant increase of fEPSP slope after NX218 application was observed compared with vehicle alone, particularly for stimulation intensities ranging from 150 to 450 μA (FIG. 1). This effect of NX218 on thalamocortical basal synaptic transmission was independent of stimulation intensity.

This result shows that NX218 peptide enhances the basal synaptic transmission between the ventrobasal thalamic nucleus and the somatosensory cortex.

### EXAMPLE 4: NX218 EFFECT ON NMDA- AND AMPA-RECEPTOR CURRENTS RECORDED ON MICE BRAIN SLICES IN MOUSE HIPPOCAMPAL CA1 NEURONS

To determine the effect of NX218 peptide on NMDA or AMPA excitatory postsynaptic currents (EPSCs), the whole cell patch-clamp method was used to perform electrophysiological recordings in control condition or during the application of NX218.

### Mice Brain Slices Preparation:

Twelve (C57Bl6/J) male mice 4-5 weeks-old were obtained from Charles River, France and housed in an animal facility. Animal care was compliant with national and local Ethics committee recommendations. Sagittal hippocampal brain slices were obtained using standard brain slicing methods (approximately 6 slices per mouse) (Knobloch et al. 2007). Mice were anesthetized with isoflurane and then decapitated. Brain was dissected out of the cranium and immediately immersed in ice-cold freshly prepared aCSF containing: 124 mM NaCl, 3.75 mM KCl, 2 mM MgSO₄, 2 mM CaCl₂, 26.5 mM NaHCO₃, 1.25 mM NaH₂PO₄, 10 mM glucose, continuously oxygenated (pH=7.4) for a total duration of 3-4 minutes. Acute slices (350 μm thick) were prepared using a vibratome (VT 1000S; Leica Microsystems, Bannockburn, IL). Sections were incubated in standard aCSF at room temperature for at least 1 h before recordings.

### Electrophysiological Recordings:

For electrophysiological recordings, a single slice was placed in the recording chamber (room temperature) submerged and continuously superfused with gassed (95% O₂, 5% CO₂) aCSF at a constant rate (2 mL·min⁻¹) for the reminder of the experiment.

In a first series of experiment, the NMDAr component of the EPSC was isolated with the addition of a GABA receptor antagonist, bicuculline (20 μM), the a-amino-3-hydroxy-5-methylisoxazole-4-propionic acid and kainic acid (AMPA/KA) receptor antagonist, 1,2,3,4-tetrahydro 6-nitro-2,3-dioxo-benzo[f]quinoxaline-7-sulfonamide (NBQX; 10 μM). For this series of experiments, a low Mg²⁺ (0.1 mM) solution was used. To keep the same extracellular divalent cation concentration, CaCl₂: 3.7 mM was included in the perfusion media.

In a second series of experiment, the AMPAr component of the EPSC was isolated with the addition of the NMDAr competitive antagonist, aminophosphoric acid (APV; 20 μM), the S)-1-(2-amino-2-carboxyethyl)-3-(2-carboxybenzyl) pyrimidine-2,4-dione kainic receptor antagonist (UBP-302; 10 μM) and a GABA receptor antagonist, bicuculline (20 μM). All chemical reagents were obtained from Alomone or Tocris Bioscience.

For somatic whole-cell recordings, patch pipettes were filled with a solution containing the following: 140 mM K-gluconate, 5 mM NaCl, 2 mM MgCl₂, 10 mM HEPES, 0.5 mM EGTA, 2 mM MgATP, 0.4 mM NaGTP, osmolarity 305 Osm/L, pH adjusted to 7.25 with KOH. The soma of large CA1 pyramidal neurons were identified and patch-clamped after visual approach of the recording pipette using a combination of infrared light and differential interference contrast optics as previously described (Jaffe and Brown 1994b; Stuart and Sakmann 1994; Stuart et al. 1993). Patch electrodes had a resistance of around 5 MΩ when filled. Recordings were terminated when the series resistances exceed 40 MΩ. The signals were digitized and low-pass filtered at 10 kHz.

EPSCs were induced in response to Schaffer collateral stimulation. For recordings of EPSCs, recordings were performed in voltage clamp at an indicated holding potential (−60 mV). The stimulation intensity was adjusted to evoke an EPSC with acceptable amplitudes.

The signal was amplified with an Axopatch 200B amplifier (Molecular Devices, Union City, CA), digitized by a Digidata 1550 interface (Molecular Devices) and sampled Clampex 10 (Molecular Devices). Recordings were acquired using Clampex (Molecular Devices) and analyzed with Clampfit (Molecular Devices). As mentioned below, one or more cells from each mouse were used and data were averaged. Experimenters were blinded to treatment groups for all experiments.

### Timeline of the Experiment and Recorded Groups:

### Series 1: Effect of NX218 Peptide on the Isolated NMDAr Currents in Hippocampal CA1 Pyramidal Neurons

A total of 10 validated neurons were included in the series 1.

In each condition, (stabilization T10 (10 minutes), NX218 peptide condition T20 (20 minutes) and wash-out T30 (30 minutes)), 10 EPSCs were induced and averaged, in response to Schaffer collateral stimulations.

### Series 2: Effect of NX218 Peptide on the Isolated AMPAr Currents in Hippocampal CA1 Pyramidal Neurons

A total of 10 validated neurons were included in the series 2.

In each condition (stabilization T10, NX218 peptide condition T20 and wash-out T30), 10 EPSCs were induced in response to Schaffer collateral stimulations. At T10, T20 and T30, 10 EPSCs were induced and averaged, in response to Schaffer collateral stimulations.

Data were analyzed by measuring the amplitudes (average of 10 EPSCs) in each condition using Clampfit (Molecular Devices):
stabilization (control)
NX218 peptide
wash-out

The data are represented as means±SEM. Using Prism 8 (Graph Pad), statistical comparisons of group means were performed to assess significant differences using RM one-way ANOVA, followed by a Tukey's post hoc test. P-value summaries: * p<0.05, ** p<0.01, *** p<0.001. Graphing of data were done with Prism 8 (Graph Pad).

### Results

The results are presented in FIGS. 2, 3, 4 and 5 and in Tables 2 and 3 below.

Examples of traces of evoked isolated NMDA and AMPA excitatory postsynaptic currents in the absence and in the presence of NX218 are presented in FIGS. 2 and 3 respectively.

Electrophysiological Evaluation of Treatment with NX218 on Evoked Isolated NMDA Excitatory Postsynaptic Currents (Series 1)

TABLE 2
Amplitude of evoked isolated NMDA excitatory postsynaptic
currents in the absence and in the presence of NX218.

| | | NX218 | |
|---|---|---|---|
| | Control | 250 μg/mL | Wash-out |
| Mean | 53.1145899 | 101.4928607 | 90.1073967 |
| SEM | 5.20352103 | 24.42031133 | 14.9938705 |
| N | 10 | 10 | 10 |

NX218 significantly increased the amplitude of evoked isolated NMDA excitatory postsynaptic currents (*, p=0.0347, RM one-way ANOVA followed by a Tukey's post hoc test). This effect is not reversed after 10 min wash-out (FIG. 4).

Electrophysiological Evaluation of Treatment with NX218 on Evoked Isolated AMPA Excitatory Postsynaptic Currents (Series 2)

TABLE 3
Amplitude of evoked isolated AMPA excitatory postsynaptic
currents in the absence and in the presence of NX218.

| | | NX218 | |
|---|---|---|---|
| | Control | 250 μg/mL | Wash-out |
| Mean | 141.690904 | 163.0687985 | 152.965846 |
| SEM | 8.16972957 | 7.392187323 | 9.2606305 |
| N | 10 | 10 | 10 |

NX218 significantly increased the amplitude of evoked isolated AMPA excitatory postsynaptic currents (*, p = 0.0226, RM one-way ANOVA followed by a Tukey's post hoc test).

This effect is not reversed after 10 min wash-out (FIG. 5).

### Conclusion

In this study, evoked NMDA- and AMPA-excitatory postsynaptic currents (EPSCs) in mouse hippocampal CA1 neurons were recorded in response to Schaffer collateral stimulation in control condition and during the application of NX218 peptide.

Application of NX218 peptide significantly increased NMDA- and AMPA-EPSCs. This increase was not reversed after 10 min wash-out.

### EXAMPLE 5: DETERMINATION OF GluN2 SUBUNIT INVOLVED IN THE EFFECT OF NX218 ON NMDA-RECEPTOR CURRENTS RECORDED IN MOUSE HIPPOCAMPAL CA1 NEURONS

To determine which GluN2 subunit was involved in the effect of NX218 increase of NMDAR excitatory postsynaptic currents (EPSCs), the whole cell patch-clamp method was used. Experiments were done in the absence or presence of NX218 at 250 μg/mL and after 10-minute wash-out. Selective antagonists of GluN2A and GluN2B subunits were also used.

### Mice Brain Slices Preparation:

Twelve (C57Bl6/J) male mice 4-5 weeks-old were obtained from Charles River, France and housed in an animal facility. Animal care was compliant with national and local Ethics committee recommendations. Sagittal hippocampal brain slices were obtained using standard brain slicing methods (approximately 6 slices per mouse) (Knobloch et al. 2007). Mice were anesthetized with isoflurane and then decapitated. Brain was dissected out of the cranium and immediately immersed in ice-cold freshly prepared aCSF containing: 124 mM NaCl, 3.75 mM KCl, 2 mM MgSO4, 2 mM CaCl2, 26.5 mM NaHCO₃, 1.25 mM NaH2PO4, 10 mM glucose, continuously oxygenated (pH=7.4) for a total duration of 3-4 minutes. Acute slices (350 μm thick) were prepared using a vibratome (VT 1000S; Leica Microsystems, Bannockburn, IL). Sections were incubated in standard aCSF at room temperature for at least 1 h before recordings.

### Electrophysiological Recordings:

For electrophysiological recordings, a single slice was placed in the recording chamber (room temperature) submerged and continuously superfused with gassed (95% O₂, 5% CO₂) aCSF at a constant rate (2 mL/min) for the reminder of the experiment.

The NMDAR component of the EPS was isolated with the addition of a GABA receptor antagonist, bicuculline (20 μM), the a-amino-3-hydroxy-5-methylisoxazole-4-propionic acid and kainic acid (AMPA/KA) receptor antagonist, 1,2,3,4-tetrahydro 6-nitro-2,3-dioxo-benzo[f]quinoxaline-7-sulfonamide (NBQX; 10 μM). A low Mg2+ (0.1 mM) solution was used. To keep the same extracellular divalent cation concentration, CaCl2 3.7 mM was included in the perfusion media.

The experiment was conducted in 3 phases as indicated below:

| | Number of cells | |
|---|---|---|
| | recorded per | |
| Experimental steps | condition | |
| 1. Phase 0: Effect of sequential application of NVP-AAM077 | | |
| and Ifenprodil on NMDA EPSCs: | | |
| (1) stabilization (control) | 3 | cells |
| (2) treatment with NVP-AAM077 (0.4 μM) | | |
| (3) treatment with Ifenprodil (3 μM) | | |
| 2. Phase 1: Evaluation of the involvement of GluN2A subunit | 10 | cells |
| in the effect of NX218 peptide observed on isolated evoked | | |
| NMDAR currents in CA1 hippocampal neurons (GluN2B | | |
| subunit were blocked with Ifenprodil): | | |
| (1) stabilization (control) | | |
| (2) treatment with Ifenprodil (3 μM) | | |
| (3) treatment with NX218 peptide (250 μg/mL) | | |
| 3. Phase 2: Evaluation of the involvement of GluN2B subunit | 10 | cells |
| in the effect of NX218 peptide observed on isolated evoked | | |
| NMDAR currents in CA1 hippocampal neurons (GluN2A were | | |
| blocked with NVP-AAM077): | | |
| (1) stabilization (control) | | |
| (2) treatment with NVP-AAM077 (0.4 μM) | | |
| (3) treatment with NX218 peptide (250 μg/mL) | | |

The phase 0 of the study aimed at showing the effect of selective sequential blockade of GluN2A and GluN2B subunits on NMDA EPSCs. First, GluN2A subunit was blocked by adding the GluN2A antagonist NVP-AAM077 (PEAQX tetrasodium hydrate) (0.4 μM) (Li et al. 2007).
NVP-AAM077 is a relatively selective GluN1/GluN2A antagonist and was shown to have more than 100-fold preferential blockade of GluN1/GluN2A vs GluN1/GluN2B (Auberson et al. 2002). Then, concomitant blockade of GluN2B subunit was achieved using the well-recognized GluN2B antagonist Ifenprodil hemitartrate (3 μM) (Li et al. 2007). Ifenprodil is one of the most selective GluN2B antagonist and has more than 200-fold preference for GluN1/GluN2B than for GluN1/GluN2A (Williams 1993).
For the phase 1 of the study (evaluation of the involvement of GluN2A subunit), the GluN2A-mediated EPSC was isolated with the addition of the GluN2B antagonist Ifenprodil (3 μM) before (T10-T20) and during (T20-T30) perfusion of NX218 peptide (250 μg/mL).
For the phase 2 of the study (evaluation of the involvement of GluN2B subunit), the GluN2B-mediated EPSC was isolated with the addition of the GluN2A antagonist NVP-AAM077 (0.4 μM) before (T10-T20) and during (T20-T30) perfusion of NX218 peptide (250 μg/mL).

All chemical reagents were obtained from Alomone Labs or Tocris Bioscience (refer to Annex 5).

For somatic whole-cell recordings, patch pipettes were filled with a solution containing the following: 140 mM K-gluconate, 5 mM NaCl, 2 mM MgCl₂, 10 mM HEPES, 0.5 mM EGTA, 2 mM MgATP, 0.4 mM NaGTP, osmolarity 305 mOsm/L, pH adjusted to 7.25 with KOH. The soma of large CA1 pyramidal neurons were identified and patch-clamped after visual approach of the recording pipette using a combination of infrared light and differential interference contrast (DIC) optics as previously described (Jaffe and Brown 1994b; Stuart and Sakmann 1994; Stuart et al. 1993). Patch electrodes had a resistance of around 5 MΩ when filled. Recordings were terminated when the series resistances exceeded 40 MΩ. The signals were digitized and low-pass filtered at 10 kHz.

Evoked postsynaptic current (EPSC) were induced in response to Schaffer collateral stimulation using a bipolar electrode. For recordings of EPSCs, experiments were performed in voltage clamp at an indicated holding potential (−60 mV). The liquid junction potential was corrected ahead of carrying out recordings.

The stimulation duration was 0.1 ms, the stimulation intensity was adjusted to evoke an EPSC with acceptable amplitudes (range of amplitude of −40 μA).

The signal was amplified with an Axopatch 200B amplifier (Molecular Devices, Union City, CA), digitized by a Digidata 1550 interface (Molecular Devices). Recordings were acquired using Clampex 10 (Molecular Devices) and analyzed with Clampfit (Molecular Devices). Two cells from each mouse were used and data were averaged. Experimenters were blinded to treatment groups for all experiments.

A total of 12 mice was dedicated to the study. A total of 23 neurons were recorded (10 per group (phases 1 and 2) and 3 in a control phase (phase 0) as follow. In each recording session, 10 EPSCs were induced in response to Schaffer collateral stimulations. Measurements of the amplitudes (average of 10 EPCS) were assessed. Phase 0 represented a validation phase aiming at confirming that GluN2A and GluN2B were the predominant NR2 subunits involved in the EPSCs evoked by Schaffer collateral stimulation. We expected the residual current at the end of phase 0 to be minimal.

### Results

The results are presented in Tables 4 and 5 below and in FIGS. 6 and 7.

### Electrophysiological Evaluation of Sequential Application of NVP-AAM077 and Ifenprodil on Evoked Isolated NMDA Excitatory Postsynaptic Currents in Hippocampal CA1 Pyramidal Neurons

NMDA EPSCs amplitude is significantly decreased by the application of NVP-AAM077 (0.4 μM) and NVP-AAM077 (0.4 μM)+Ifenprodil (3 μM) (data not shown).

Evaluation of the Involvement of GluN2A Subunit in the Effect of NX218 Peptide Observed on Isolated Evoked NMDAR Currents in CA1 Hippocampal Neurons (GluN2B Subunits were Blocked with Ifenprodil)

TABLE 4
Normalized amplitude of evoked isolated NMDA excitatory
postsynaptic currents before (Control) and after
sequential addition of Ifenprodil (3 μM) (GluN2B
antagonist) and NX218 peptide (250 μg/mL).

| | | | Ifenprodil + NX218 |
|---|---|---|---|
| | Control | Ifenprodil | 250 μg/mL |
| Mean | 100 | 78.74 | 90.55 |
| SEM | | 2.78 | 4.20 |
| N | 10 | 10 | 10 |

Evaluation of the Involvement of GluN2B Subunit in the Effect of NX218 Peptide Observed on Isolated Evoked NMDAR Currents in CA1 Hippocampal Neurons (GluN2A Subunits were Blocked with NVP-AAM077)

TABLE 5
Normalized amplitude of evoked isolated NMDA excitatory
postsynaptic currents before (Control) and after
sequential addition of NVP-AAM077 (0.4 μM) (GluN2B
antagonist) and NX218 peptide (250 μg/mL).

| | | NVP- | NVP-AAM077 + NX218 |
|---|---|---|---|
| | Control | AAM077 | 250 μg/mL |
| Mean | 100 | 56.35 | 63.69 |
| SEM | | 2.94 | 4.39 |
| N | 10 | 10 | 10 |

### Conclusion

In this study, pharmacological tools were used to isolate postsynaptic currents mediated by GluN2A- and GluN2B-containing NMDARs. Isolated evoked NMDAR currents were recorded using the whole-cell patch-clamp method in mouse hippocampal CA1 neurons in response to Schaffer collateral stimulation in control condition and during the application of NX218 peptide at 250 μg/mL with selective antagonists of GluN2A and GluN2B subunits. The aim was to determine which GluN2 subunit was involved in the effect of NX218 increase of NMDAR EPSC currents.

In this study, we observed:
A significant decrease of NMDA EPSCs amplitude after sequential application of NVP-AAM077 (0.4 μM) and Ifenprodil (3 μM), showing that the excitatory postsynaptic current is predominantly mediated by ionotropic glutamate receptors.
Addition of NX218 (250 μg/mL) after blockade of GluN2B subunit by Ifenprodil (3 μM) significantly increased NMDA excitatory postsynaptic currents. Therefore, GluN2A subunit is involved in the effect of NX218 increase of NMDAR EPSC currents.
Addition of NX218 (250 μg/mL) after blockade of GluN2A subunit by NVP-AAM077 (0.4 μM) had no significant effect on NMDAR EPSC currents. Therefore, GluN2B subunit is not involved in the effect of NX218 increase of NMDAR EPSC currents.

### EXAMPLE 6: EVALUATION OF THE EFFECT OF NX210 AND NX218 IN THE SCOPOLAMINE MOUSE MODEL OF AMNESIA

To evaluate the effect of NX210 and its cyclic version NX218 against scopolamine-induced learning and memory impairments, the Y-Maze cognitive test was used in the Scopolamine mouse model of amnesia.

### Methods

Male Swiss mice, weighing 30-35 g, from JANVIER (Saint Berthevin, France), were housed in groups with access to food and water ad libitum, except during behavioral experiments. They were kept in a temperature and humidity-controlled animal facility on a 12 h/12 h light/dark cycle (lights off at 07:00 μm). Mice were numbered by marking their tail with permanent markers. All animal procedures were conducted in strict adherence to the European Union directive of Sep. 22, 2010 (2010/63/UE). In each cage (n=6 per cage), treatment regimen was the same. Animals were tested in a random and blind manner.

NX210 and NX218 compounds were solubilized in water for injection (vehicle) and administered intraperitoneally 1, 2, 24 or 48 hours before the Y-maze (YM) test.

Donepezil (DPZ) used as positive control was administered per os at a dose of 1 mg/kg once 1 h before the YM test.

Scopolamine was administered at a dose of 0.5 mg/kg by subcutaneous injection 30 minutes before the YM session.

After administration of compounds at different timepoints all animals were tested for spontaneous alternation performance in the YM, an index of spatial working memory. The YM was designed according to Itoh and collaborators (1993) and Hiramatsu and Inoue (1999) and is made of grey polyvinylchloride. Each arm is 40-cm long, 13-cm high, 3-cm wide at the bottom, 10-cm wide at the top, and converging at an equal angle. Each mouse was placed at the end of one arm and allowed to move freely through the maze during an 8-min session. The series of arm entries, including possible returns into the same arm, were checked visually. An alternation was defined as entries into all three arms on consecutive occasions. The number of maximum alternations is therefore the total number of arm entries minus two and the percentage of alternation was calculated as (actual alternations/maximum alternations)×100. Parameters included the percentage of alternation, called memory index, and total number of arm entries, called exploration index (Maurice et al., 1996, 1988; Meunier et al., 2006; Villard et al., 2009, 2011).

Animals showing an extreme behavior (alternation percentage <20% or >90% or number of arm entries <10) would have been discarded from the calculation. No animal was discarded accordingly.

### Results

The results of NX210 or NX218 administration (at a dosage of 10, 15 or 20 mg/kg) 24 hours before the YM test are presented in Table 6 and FIG. 8. Values are spontaneous alternance %.

TABLE 6
Effect of intraperitoneal administration of NX210 or NX218 on scopolamine-induced
spatial short-term working memory deficits in mice using the YM cognitive test.
NX210 or NX218 were administered at 3 doses (10, 15 and 20 mg/kg) 24 h before the
test. Donepezil (DPZ) was administered 1 h before the test (positive control).

| | | | | Scopolamine + | Scopolamine + |
|---|---|---|---|---|---|
| | | | Scopolamine + | NX210 | NX210 |
| | Control | Scopolamine | Donepezil | 10 mg/kg -24 h | 15 mg/kg - 24 h |
| Mean | 70 | 45 | 73 | 59 | 69 |
| SEM | 2.1 | 2.8 | 2.7 | 1.9 | 3.1 |
| N | 6 | 6 | 6 | 6 | 6 |

| | Scopolamine + | Scopolamine + | Scopolamine + | Scopolamine + |
|---|---|---|---|---|
| | NX210 | NX218 | NX218 | NX218 |
| | 20 mg/kg -24 h | 10 mg/kg -24 h | 15 mg/kg -24 h | 20 mg/kg - 24 h |
| Mean | 63 | 71 | 55 | 59 |
| SEM | 3.1 | 2.1 | 2 | 2.6 |
| N | 6 | 6 | 6 | 6 |

### Conclusion

Scopolamine is a cholinergic blocker that induces amnesic effect on spatial short-term working memory as highlighted in the YM.

Donepezil, used as a positive control, significantly reversed the deficits in terms of alternation in the YM when administered 1 h before the behavioral test.

When administered once 24 h before the YM test (24 h pre-treatment) at different doses, NX210 and NX218 peptides showed a dose-response effect on preventing spatial short-term working memory deficits induced by the acute scopolamine administration.

The beneficial effects of test compound NX210 were demonstrated from the dose of 10 mg/kg, with the maximum reversal effect on spatial short-term working memory impairments displayed at 15 mg/kg, and then decreased but still significative at 20 mg/kg (bell-shaped curve). No effect was displayed when the dose of NX210 was lower than 10 mg/kg or when administered 2 h or 1 h before the YM test (data not shown). When administered once 48 h before the YM test, the positive effects of NX210 were maintained but less pronounced (data not shown), and only at the highest dose (15 mg/kg).

Test compound NX218 at 5 mg/kg showed a complete reversal effect on spatial short-term working memory impairments when administered 24 h before the YM test (data not shown). This restoration was only partial when administered 2 h before the test, and null when administered 1 h prior to the YM test (data not shown). When administered 24 h before this cognitive test, the efficacy was already demonstrated at the lowest tested dose, i.e. 2.5 mg/kg, with a maximum effect observed at 10 mg/kg. The two highest doses, 15 and 20 mg/kg, only partially blocked the scopolamine induced memory deficits as displayed by the YM test (bell-shaped dose-response curve). When administered 48 h before the test, NX218 at 5 and 15 mg/kg partially blocked the scopolamine effect on the YM test, whereas the 10 mg/kg dose completely counteracted the scopolamine induced short-term memory deficits (data not shown).

### EXAMPLE 7: EVALUATION OF THE EFFECT OF NX218 ON FUNCTIONAL RECOVERY IN AN IN VITRO MODEL OF HYPOXIA

Acute episodes of hypoxia may cause a depression in synaptic activity in many brain regions. In the preceding examples, we showed that NX218 (250 μg/mL) acts through GluN2A subunits to increase NMDA-mediated current amplitudes in CA1 hippocampal neurons after Schaffer collateral stimulation. The aim of this study was to determine if the NX218 peptide can improve functional recovery in an in vitro model of hypoxia (Hédou et al., 2008; Farinelli et al., 2012). To meet this goal, functional recovery was assessed by recording evoked field excitatory postsynaptic potentials (fEPSPs) in mouse hippocampal neurons, before and after oxygen-glucose deprivation (OGD). To evaluate the effect of NX218, experiments were done on different slices, superfused with NX218 or vehicle.

### Experimental Design:

| | Number of slices |
|---|---|
| Group treatments and experimental steps | per condition |
| 1. Measurement of the functional recovery following | 9 |
| OGD (control slices) | |
| (1) baseline (10 min) | |
| (2) OGD induction (sucrose-aCSF, 95% N₂, 5% CO₂) + | |
| treatment with vehicle (WFI) | |
| (3) recovery (return to normoxic conditions) | |
| 2. Evaluation of the effect of NX218 on functional | 9 |
| recovery following OGD (treated slices) | |
| (1) baseline (10 min) | |
| (2) OGD induction (sucrose-aCSF, 95% N₂, 5% CO₂) + | |
| treatment with NX218 (250 μg/mL) | |
| (3) recovery (return to normoxic conditions) | |
| Total number of slices | 18 |

### Methods

### Animals

Experiments were carried out with 4-5 weeks old male C57Bl6/J mice (approximately 20 grams) obtained from Charles River, France. In total, 9 mice were used in this study. Two slices were recorded from each mouse (one for the control condition and another for the NX218 condition). Animals were acclimated to laboratory housing conditions for 13 days prior to experimental use. Standard Food (Type a04, SAFE, France) was available ad libitum. Filtered mains drinking water (0.22 μM) was available ad libitum.

### Mice Brain Slices Preparation

Sagittal hippocampal brain slices were obtained using standard brain slicing methods (approximately 6 slices per mouse) (Knobloch et al. 2007). Mice were anesthetized with 5% isoflurane and then decapitated. Brain was dissected out of the cranium and immediately immersed in ice-cold freshly prepared artificial cerebrospinal fluid (aCSF) containing: 124 mM NaCl, 3.75 mM KCl, 2 mM MgSO₄, 2 mM CaCl₂, 26.5 mM NaHCO₃, 1.25 mM NaH₂PO₄, 10 mM glucose, continuously oxygenated (95% O2, 5% CO2) (pH=7.4) for a total duration of 3-4 minutes. Acute slices (350 μm thick) were prepared using a vibratome (VT 1000S; Leica Microsystems, Bannockburn, IL). Sections were incubated in standard aCSF (124 mM NaCl, 3.75 mM KCl, 2 mM MgSO4, 2 mM CaCl2, 26.5 mM NaHCO₃, 1.25 mM NaH2PO4, 10 mM glucose) at room temperature for at least 1 h before recordings.

### Electrophysiological Recordings

For electrophysiological recordings, a single slice was placed in the recording chamber (room temperature), submerged and continuously superfused with gassed aCSF (95% O2, 5% CO2; pH=7.4) at a constant rate (2 mL min⁻¹) for the reminder of the experiment. Extracellular field excitatory postsynaptic potentials (fEPSPs) were recorded in the CA1 stratum radiatum using a glass micropipette filled with aCSF. fEPSPs were evoked by the electrical stimulation of Schaffer collateral-commissural pathway at 0.1 Hz (i.e., a single pulse every 10 s) with a glass stimulating electrode (borosilicate capillary glass with filament, standard wall; OD: 1.5 mm; ID: 0.86 mm; Length: 75 mm; ref: W3 30-0060 from Harvard Apparatus) placed in the stratum radiatum.

At the beginning of each experiment (when a stable fEPSP response was reached), Input/Output (I/O) curves were obtained by gradual increases in stimulus intensity (from 0 to 100 μA, 10 μA intervals) to determine the intensity of stimulation that was kept for the reminder of the experiment. Stable baseline fEPSPs was then recorded during 10 minutes by stimulating at 80% of maximal field amplitude (single pulse every 10 s, i.e., 0.1 Hz).

Recordings were performed before and after OGD (10-15 min) in mouse hippocampal slices superfused with vehicle (WFI) or NX218 at 250 μg/mL. Measurements of evoked field excitatory postsynaptic potential (fEPSP) recovery after oxygen-glucose deprivation (OGD) were continued for 90 min from the start of OGD induction.

Signals were amplified with an Axopatch 200B amplifier (Molecular Devices, Union City, CA) digitized by a Digidata 1322A interface (Axon Instruments, Molecular Devices, US) and sampled at 10 kHz. Recordings were acquired using Clampex (Molecular Devices) and analyzed with Clampfit (Molecular Devices).

Experimenters were blinded to treatment for all experiments. To ensure rigorous comparability of data in both groups, two slices were used from each mouse: one was used as a control slice, and another was used as a treated slice.

### Oxygen-Glucose Deprivation

After a 10 min stable baseline, acute slices were exposed to 10-15 min of hypoxic/aglycemic conditions by perfusion with sucrose aCSF depleted of glucose (0 mM glucose, replaced with equimolar sucrose) and gassed with 95% N₂, 5% CO₂. OGD was applied in slices until the f-EPSP slope reached a value that represented at least less than 10% of the averaged baseline value. At this time point, we considered that the effect of OGD is induced. In this study, the mean OGD duration was 9.6±2.8 min.

### Data Processing and Statistical Analysis

Data were analyzed by measuring the amplitudes of f-EPSPs in each condition using Clampfit (Molecular Devices). Using Prism 8 (Graph Pad), 2-way-ANOVA statistical test followed by a Šidák's multiple comparisons test was performed to assess significant differences. P-value summaries: * p<0.05, ** p<0.01, *** p<0.001. All data are presented as mean±SEM (standard error of the mean), except for OGD mean duration which is expressed as mean±SD (standard deviation).

### Results

### Electrophysiological Evaluation of the Effect of NX218 on Functional Recovery in an In Vitro Model of Hypoxia in Mice:

The results are presented in FIG. 9, Table 7 and FIG. 10.

FIG. 9: Time course of the averaged normalized f-EPSP slope evoked by Schaffer collateral stimulation during and after oxygen/glucose deprivation (OGD) in mouse hippocampal slices superfused with vehicle (n=9) or NX218 at 250 μg/mL (n=9). Black circles represent the response in vehicle-treated slices (control slices, n=9) before, during and after OGD induction. Gray triangles represent the response in NX218-treated slices (treated slices, n=9) before, during and after OGD induction.

TABLE 7
Effect of the perfusion of NX218 on the averaged normalized
f-EPSP slope before (baseline) and different
timepoints after the start of OGD induction.

| | | Base- | 0-30 | 30-60 | 60-90 | last | |
|---|---|---|---|---|---|---|---|
| | | line | min | min | min | 10 min | n= |
| vehicle | Mean | 100 | 31.618 | 47.152 | 69.598 | 72.432 | 9 |
| | SEM | | 2.893 | 4.483 | 3.482 | 2.861 | |
| NX218 | Mean | 100 | 31.043 | 61.663 | 87.959 | 92.639 | 9 |
| | SEM | | 3.432 | 4.959 | 2.652 | 2.378 | |

FIG. 10: Bar graphs of the averaged normalized f-EPSP slope evoked by Schaffer collateral stimulation during and after oxygen/glucose deprivation (OGD) in mouse hippocampal slices superfused with vehicle (n=9) or NX218 at 250 μg/mL (n=9). White bars represent f-EPSP slopes in vehicle-treated slices (control slices, n=9) before (baseline) and different timepoints after the start of OGD induction. Hatched bars represent f-EPSP slopes in NX218-treated slices (treated slices, n=9) before (baseline) and different timepoints after the start of OGD induction. P-value summaries: * p<0.05, ** p<0.01, *** p<0.001.

### Conclusion

In this study, we observed that functional recovery following acute hypoxia in vitro is improved by NX218 treatment. Indeed, a substantial synaptic depression (i.e. depression of fEPSP slope) at Schaffer collateral-CA1 synapses occurred following OGD induction in hippocampal slices. Staggeringly, treatment with NX218 (250 μg/mL) statistically increase the recovery after OGD in hippocampal slices, at different timepoints after the start of OGD induction: fEPSP slopes were significantly higher in NX218-treated slices than in vehicle-treated slices as early as 30-60 minutes after OGD induction (fEPSP slope expressed as a % of baseline: 61.7±5.0 for NX218 versus 47.1±4.5 for vehicle; *; p=0.045). This beneficial effect was even more striking at 60-90 minutes (88.0±2.6 for NX218 versus 69.6±3.5 for vehicle; ***; p<0.001) and persisted until the last 10 minutes post-OGD induction (92.6±2.4 for NX218 versus 72.4±2.9 for vehicle; ***; p<0.001).

### EXAMPLE 8: EVALUATION OF THE EFFECT OF NX218 ON FUNCTIONAL RECOVERY IN AN IN VITRO MODEL OF HYPOXIA

In an in vitro model of hypoxia (Hédou et al., 2008; Farinelli et al., 2012), we previously showed in Example 7 that NX218 (250 μg/mL) promotes recovery of synaptic transmission when added from the start of Oxygen/Glucose Deprivation (OGD). The aim of this study was to determine whether this beneficial effect on post-hypoxia recovery could be maintained even if NX218 was added somewhat later (after the end of OG). Two extra slices were also be recorded to evaluate a possible curative capacity of NX218 against hypoxia. To this end, NX218 was added during the recovery plateau following OG, more precisely at T=60 minutes and T=70 minutes after OG initiation.

### Experimental Design

| | Number of slices |
|---|---|
| Group treatments and experimental steps | per condition |
| 1. Measurement of the functional recovery following | 10 |
| OGD (control slices) | |
| (1) baseline (10 min) | |
| (2) OGD induction (sucrose-aCSF, 95% N₂, 5% CO₂) | |
| (3) treatment with vehicle (WFI) + recovery (return to | |
| normoxic conditions) | |
| 2. Evaluation of the effect of NX218 on functional | 10 |
| recovery following OGD (treated slices) | |
| (1) baseline (10 min) | |
| (2) OGD induction (sucrose-aCSF, 95% N₂, 5% CO₂) | |
| (3) treatment with NX218 (250 μg/mL) + recovery | |
| (return to normoxic conditions) | |
| 3. Evaluation of the curative effect of NX218 on | 2 |
| neuronal activity following OGD (“test slices”) | |
| (1) baseline (10 min) | |
| (2) OGD induction (sucrose-aCSF, 95% N₂, 5% CO2) | |
| (3) treatment with vehicle (WFI) + recovery (return to | |
| normoxic conditions) | |
| (4) treatment with NX218 (250 μg/mL) during recovery | |
| plateau (T = 60 min or T = 70 min post OGD induction) | |
| Total number of slices | 22 |

### Methods

Animals: see Example 7. In total, 10 mice were used in this study. An additional mouse was used to obtain two extra slices for preliminary recordings (“test slices)”.

Mice brain slice preparation: see Example 7

### Electrophysiological Recordings:

Conditions of Example 7 are used, except:

Each slice was superfused with either vehicle (WFI) or NX218 at 250 μg/mL from the end of OGD and until the end of the experiment.

Measurements of evoked field excitatory postsynaptic potential (fEPSP) recovery after OGD were continued for 120 min from TO (OGD initiation). Two “test slices” were also done according to the experimental design described above.

Oxygen-glucose deprivation: see Example 7. In this study, the mean OGD duration was 10.6±1.0 min.
Data processing and statistical analysis: see Example 7.

### Results

### Electrophysiological Evaluation of the Effect of NX218 on Functional Recovery in an In Vitro Model of Hypoxia in Mice:

The results are presented in FIG. 11, Table 8 and FIG. 12.

FIG. 11: Time course of the averaged normalized f-EPSP slope evoked by Schaffer collateral stimulation during and after oxygen/glucose deprivation (OGD) in mouse hippocampal slices superfused with vehicle (n=10) or NX218 at 250 μg/mL (n=10) from the end of OGD. Black circles represent the response in vehicle-treated slices (control slices, n=10) before, during and after OGD induction. Gray triangles represent the response in NX218-treated slices (treated slices, n=10) before, during and after OGD induction.

TABLE 8
Effect of a more delayed perfusion of NX218 on the averaged normalized f-EPSP
slope before (baseline) and different timepoints after the start of OGD induction.

| | | Baseline | 0-30 min | 30-60 min | 60-90 min | 90-120 min | last 10 min | n= |
|---|---|---|---|---|---|---|---|---|
| vehicle | Mean | 100 | 28.497 | 53.456 | 68.774 | 77.110 | 79.319 | 10 |
| | SEM | | 3.177 | 7.665 | 5.509 | 2.694 | 2.707 | |
| NX218 | Mean | 100 | 25.758 | 52.556 | 74.616 | 88.046 | 90.340 | 10 |
| | SEM | | 2.167 | 6.503 | 4.682 | 1.934 | 2.674 | |

FIG. 12: Bar graph of the averaged normalized f-EPSP slope evoked by Schaffer collateral stimulation during and after oxygen/glucose deprivation (OGD) in mouse hippocampal slices superfused with vehicle (n=10) or NX218 at 250 μg/mL (n=10) at the end of OGD. White bars represent f-EPSP slopes in vehicle-treated slices (control slices, n=10) before (baseline) and different timepoints after the start of OGD induction. Hatched bars represent f-EPSP slopes in NX218-treated slices (treated slices, n=10) before (baseline) and different timepoints after the start of OGD induction. P-value summaries: * p<0.05, ** p<0.01, *** p<0.001.

Evaluation of the Curative Effect of NX218 on Neuronal Activity Following OGD (Preliminary Sample Study Using a Single Slice Recording; n=2):

The results are presented in FIG. 13.

FIG. 13: Time course of the averaged normalized f-EPSP slope evoked by Schaffer collateral stimulation during and after oxygen/glucose deprivation (OGD) in mouse hippocampal slices superfused with NX218 at 250 μg/mL during recovery plateau, at T=60 min and T=70 min after OGD initiation (n=1 per timepoint).

### Conclusion

Here we observed that functional recovery following acute hypoxia in vitro is improved by NX218, even with a more delayed perfusion (hypoxic/aglycemic conditions, mean duration=10.6 min; NX218 perfusion from the end of OGD). Indeed, treatment with NX218 (250 μg/mL) at the end of OGD statistically increase the recovery of hippocampal neuronal activity since T=90 minutes (88.0±1.9 for NX218 versus 77.1±2.7 for vehicle; **; p=0.004) and persisted until the end of the experiment at 120 minutes post OGD initiation (90.3±2.7 for NX218 versus 79.3±2.7 for vehicle; **, p=0.01).

Two extra slices were done to evaluate the curative capacity of NX218 against hypoxia. The application NX218 at 250 μg/mL during recovery plateau (T=60 min or T=70 min) following OGD modified the shape of the recovery phase by rising further toward baseline. This preliminary study sheds light on the potential curative effect of NX218 after hypoxia.

### EXAMPLE 9: NX218 EFFECT ON BASAL HIPPOCAMPAL SYNAPTIC TRANSMISSION MEASURED ON MICE BRAIN SLICES

To determine the effect of NX218 peptide on basal excitatory synaptic transmission in the CA1 region of mice hippocampi, Extracellular field excitatory postsynaptic potentials (fEPSPs) were recorded in response to Schaffer collateral stimulation on mice hippocampal slices superfused with vehicle then with NX218 at 250 μg/mL. Comparison of postsynaptic responses in control versus NX218 peptide conditions were assessed.

### Mice Brain Slices Preparation:

Five (C57Bl6/J) male mice 4-5 weeks-old were obtained from Charles River, France and housed in an animal facility. Animal care was compliant with national and local Ethics committee recommendations. Ten Sagittal hippocampal brain slices (2 per mouse) were obtained using standard brain slicing methods (Knobloch et al. 2007). Mice were anesthetized with 5% isoflurane and then decapitated. Brain was dissected out of the cranium and immediately immersed in ice-cold freshly prepared artificial cerebrospinal fluid (aCSF) containing: 124 mM NaCl, 3.75 mM KCl, 2 mM MgSO₄, 2 mM CaCl₂, 26.5 mM NaHCO₃, 1.25 mM NaH₂PO₄, 10 mM glucose, continuously oxygenated (95% O₂, 5% CO₂) (pH=7.4) for a total duration of 3-4 minutes. Acute slices (350 μm thick) were prepared using a vibratome (VT 1000S; Leica Microsystems, Bannockburn, IL). Sections were incubated in standard aCSF (124 mM NaCl, 3.75 mM KCl, 2 mM MgSO₄, 2 mM CaCl₂, 26.5 mM NaHCO₃, 1.25 mM NaH₂PO₄, 10 mM glucose) at room temperature for at least 1 h before recordings.

### Electrophysiological Recordings:

For electrophysiological recordings, a single slice was placed in the recording chamber (room temperature), submerged and continuously superfused with gassed aCSF (95% O₂, 5% CO₂; pH=7.4) at a constant rate (2 mL min⁻¹) for the reminder of the experiment. Extracellular field excitatory postsynaptic potentials (fEPSPs) were recorded in the CA1 stratum radiatum using a glass micropipette filled with aCSF. fEPSPs were evoked by the electrical stimulation of Schaffer collateral-commissural pathway at 0.1 Hz (i.e., a single pulse every 10 s) with a glass stimulating electrode (borosilicate capillary glass with filament, standard wall; OD: 1.5 mm; ID: 0.86 mm; Length: 75 mm; ref: W3 30-0060 from Harvard Apparatus) placed in the stratum radiatum.

After 10 minutes of stabilization, basal synaptic transmission was assessed. To this purpose, gradual increases in stimulus intensity, from 0 to 850 μA, 50 μA increments, at 0.1 Hz (i.e. a single pulse every 10 s), was delivered from the stimulating electrode and fEPSPs slopes responses from the recording electrode was quantified. This procedure was repeated 3 times, 30 seconds interval for each session. A single session final I/O curve was obtained by plotting the averaged rationalised slopes of fEPSPs as a function of the stimulation intensity. Sessions occured respectively:
A: after a 10-minute perfusion of vehicle, slices being still superfused with vehicle during I/O recordings.
B: after a 10-minute perfusion of NX218, slices being still superfused with NX218 during I/O recordings.

The signal was amplified with an Axopatch 200B amplifier (Molecular Devices, Union City, CA) digitized by a Digidata 1322A interface (Axon Instruments, Molecular Devices, US) and sampled at 10 kHz. Recordings were acquired using Clampex (Molecular Devices) and analyzed with Clampfit (Molecular Devices).

### Results

All data was normalized from the fEPSP maximum slope recorded in control condition (during vehicle bath application). The results obtained for the NX218 condition were thus normalized to control condition (vehicle) this way. Potential outliers were identified for exclusion using the following criteria: >3 standard deviations away from the group mean in either direction. The results from 10 validated slices are presented in Table 9 and in FIG. 14.

TABLE 9
Normalized average slope of fEPSP evoked during input-output (I/O) recordings
in the CA1 area following Schaffer collateral stimulation in hippocampal slices sequentially
superfused with vehicle and NX218 at 250 μg/mL.

| | | Stimulation intensity(pA) | | | | | | | | |
|---|---|---|---|---|---|---|---|---|---|---|
| | | 0 | 50 | 100 | 150 | 200 | 250 | 300 | 350 | 400 |
| Normalized | Mean | −0.20 | 18.46 | 42.72 | 65.33 | 78.64 | 87.59 | 93.61 | 94.72 | 96.34 |
| fEPSP | Control | | | | | | | | | |
| slope | Mean NX218 | 0.09 | 30.78 | 71.26 | 110.51 | 134.44 | 149.89 | 156.80 | 160.72 | 164.01 |
| (%) | SEM Control | 0.34 | 2.29 | 3.39 | 4.13 | 3.33 | 1.54 | 1.00 | 0.78 | 0.73 |
| | SEM NX218 | 0.15 | 6.17 | 10.85 | 15.57 | 18.02 | 18.93 | 18.72 | 18.76 | 19.36 |
| | | Stimulation intensity(pA) | | | | | | | | |
| | | 450 | 500 | 550 | 600 | 650 | 700 | 750 | 800 | 850 |
| Normalized | Mean | 96.75 | 97.67 | 97.64 | 97.32 | 98.48 | 97.68 | 97.58 | 97.64 | 97.60 |
| fEPSP | Control | | | | | | | | | |
| slope | Mean NX218 | 164.39 | 166.29 | 167.00 | 167.37 | 168.66 | 169.65 | 169.89 | 170.46 | 171.65 |
| (%) | SEM Control | 0.58 | 0.44 | 0.49 | 0.81 | 0.81 | 0.79 | 1.15 | 1.21 | 1.47 |
| | SEM NX218 | 19.60 | 20.09 | 19.89 | 20.07 | 20.41 | 20.36 | 20.58 | 20.86 | 21.00 |

In this study, a statistical increase of the basal excitatory synaptic transmission at CA3-CA1 synapses in mouse hippocampal slices superfused with NX218 was observed, when compared to control condition.

This result shows that NX218 peptide enhances the basal hippocampal synaptic transmission.

### EXAMPLE 10: EFFECT OF NX218 ON SUBCHRONIC PCP-INDUCED COGNITIVE DEFICITS IN MICE

Phenycyclidine (PCP) is an antagonist of NMDA receptor, its administration in healthy humans produces schizophrenia-like symptoms, therefore PCP is often used to mimic schizophrenia in rodents. To evaluate the effect of NX218 against subchronic PCP-induced cognitive deficits in mice, the T-Maze continuous alternation task was used. In addition, brain samples were collected shorlty after the T-Maze test to measure the levels of cerebral biomarkers involved in synaptic plasticity.

### Methods

### Animals:

Sixty (60) male Swiss CD-1 mice were purchased from Janvier Labs (Le Genest-Saint-Isle, France) one week before the start of the study for acclimation. Mice were group-housed (6-8 mice per Eurostandard type Ill cage) and maintained in a room with controlled temperature (21-22° C.) under a reversed 12 h/12 h light-dark cycle (lights on: 5:30 μm; lights off: 05:30 am) with food and water available ad libitum. The weight of mice ranged from 25.9 and 36.4 g.

Health of animals was checked daily. General aspect of animals and their activity were inspected daily whereas weight was monitored before PCP and peptide administration. All animal procedures were performed in compliance with the existing legislation and regulations (European Directive 2010/63/EU incorporated in French law, amended by Decree No. 2013-118 of 18 Feb. 2013).

### Compounds Administration and Treatment Groups Description:

PCP (or saline for control group) was administered at a dose of 0.2 mg/kg by subcutaneous injections twice daily for 12 days (day 0 to day 11).

NX218 was solubilized in water for injection (vehicle) and administered intraperitoneally at a dose of 5 mg/kg 48 h, 24 h and/or 2 h prior to the T-maze trial.

Nicotine used as a positive control was administered at a dose of 0.4 mg/kg intraperitoneally 30 minutes prior to the T-maze trial

| | Dose | | Concentration | Animal |
|---|---|---|---|---|
| Group description | level | | in mg/mL | (n) |
| Vehicle (D12-D14)/Saline | NA | | 0 | 10 |
| Vehicle (D12-D14)/PCP (0.2 mg/kg) | NA | | 0 | 10 |
| Nicotine (D14)/PCP (0.2 mg/kg) | 0.4 | mg/kg | 0.04 | 10 |
| NX218 (D14)/PCP (0.2 mg/kg) | 5 | mg/kg | 0.5 | 10 |
| NX218 (D13)/PCP (0.2 mg/kg) | 5 | mg/kg | 0.5 | 10 |
| NX218 (D12-D13-D14)/PCP (0.2 mg/kg) | 5 | mg/kg | 0.5 | 10 |

### Experimental Procedure:

After administration of compounds at different timepoints all animals were tested for continuous alternation task in the T-Maze. The T-maze apparatus was made of gray Plexiglas with a main stem (55-cm long×10-cm wide×20-cm high) and two arms (30-cm long×10-cm wide×20-cm high) positioned at a 90-degree angle relative to the main stem. A start box (15-cm long×10-cm wide) was separated from the main stem by a guillotine door. Horizontal doors were also provided to close specific arms during the force choice alternation task. The experimental protocol consisted in one single session that started with 1 “forced-choice” trial, followed by 14 “free-choice” trials (Gerlai, 1998). In the first “forced-choice” trial, the mouse was confined 5 s in the start arm and then released while either the left or right goal arm was blocked by closing the sliding door. Then, it walked through the maze, eventually entered the open goal arm, and then returned to the start position. Immediately after the mouse came back to the start position, the left or right goal door was opened (i.e. the two goal doors were opened), and the mouse could choose freely between the left and right goal arms (first “free choice trial”). The mouse was considered as entered in the arm when it placed its four paws in the arm. The opposite goal door was then closed until the animal returned to the start arm. Then, a second “free choice trial” began by the opening of the closed goal door (i.e. the two goal doors were opened), leaving the choice to the mouse to enter either into the right or the left arm. The session was considered finished as soon as 14 free-choice trials had been performed in less than 10 min, and the mouse was removed from the maze. The time needed to reach these 14 “free-choices” was recorded. None of the mice spent more than 10 minutes to perform the 14 choices, which would have been an exclusion criterion.

The percent of spontaneous alternations was calculated as number of spontaneous alternations divided by the number of free-choice trials, an index of spatial short-term working memory. The apparatus was cleaned between each animal using alcohol (70%). Urine and feces were removed from the maze. During the trials, animal handling and the visibility of the operator were minimized as much as possible. Animals were tested in a random and blind manner.

### Brain Sampling:

Approximately 5 minutes after the test, mice were anaesthetized with 5% isoflurane oxygen mixture. Brains were extracted immediately, then the cortex of each hemisphere were collected. Right and left cortices were transferred into clean pre-labelled microtubes and the exact weight of each sample was recorded. Finally, samples were stored at −80° C. for further analysis.

### Protein Extraction and Automated Analysis:

Brain samples were lysed with a defined buffer lysis consisting of CelLyticMT reagent with 1% of protease and phosphatase inhibitor cocktail (60 μL per well). Lysates were processed at +4° C. and stored at −80° C. at the end of the experiment. For each sample, the quantity of proteins was determined using the micro kit BCA (Pierce). Briefly, lysates were centrifuged and diluted at 1/20 in PBS and mixed, in equal volume, with a micro BCA. After an incubation at 60° C. for 1 hour, the quantity of proteins was measured at 562 nm with a spectrophotometer Nanovue (GE Healthcare) and compared with the standard of Bovine Serum Albumin curve (BSA, Pierce).

Automated protein analysis were performed using the WES™ system (ProteinSimple®):

Reagents were prepared and used according to manufacturer's recommendations for use on WES™ (ProteinSimple, https://www.proteinsimple.com/wes.html).

The run was performed according to manufacturer's recommendations. Capillaries, samples (3 μL of a solution at 0.2 mg/mL for GluN2A and 2 mg/mL for phosphorylated CREB), antibodies and matrices were then loaded inside the instrument. The simple Western was run with capillaries filled with separation matrix, stacking matrix and protein samples. Then, capillaries were incubated 2 h with primary antibodies, at room temperature (23° C., ±3° C.):
anti-pCREB antibody (Cell signaling, 9198S, 43 kDa)
anti-total-GluN2A antibody (Millipore Sigma, M264-10UG, ˜180 kDa)

Each protein was evaluated independently. Capillaries were washed and incubated with highly sensitive horseradish peroxidase (HRP) conjugated secondary antibodies for 1 h, at room temperature (23° C., 3° C.). After removal of unbound secondary antibody, the capillaries were incubated at room temperature (23° C., 3° C.), with the luminol-S/peroxide substrate and chemiluminescent signal was collected using the Charge-Coupled Device (CCD) camera of WES™ with six different exposure times (30, 60, 120, 240, 480, and 960 s). Data analysis was performed using the Compass Software (ProteinSimple) on WES™. 5 samples (biological replicates) per condition were analyzed.

### Results

### T-Maze Assay:

The results of NX218 administration on subchronic PCP-induced cognitive deficit are presented in table 10 and FIG. 15.

TABLE 10
Effect of intraperitoneal administration of NX218 on subchronic PCP-
induced cognitive deficits in mice using T-maze cognitive test.

| | Spontaneous | | |
|---|---|---|---|
| | alternations (%) | | |
| Group | Mean | SEM | N |
| Control | 66.43 | 3.20 | 10 |
| PCP D1-D11 | 30.71 | 2.62 | 10 |
| PCP + Nicotine | 62.14 | 1.86 | 10 |
| PCP + NX218 5 mg/kg D14 | 52.87 | 4.01 | 10 |
| PCP + NX218 5 mg/kg D13 | 34.92 | 1.43 | 9 |
| PCP + NX218 5 mg/kg D12/D13/14 | 62.13 | 2.14 | 10 |

Subchronic PCP-treated mice scored 31% spontaneous and continuous alternation performance in the T-maze. This performance was significantly different from the saline-treated mice (66%) and reflects a subchronic PCP-induced deficit in this task.

2 h pretreatment with NX218 on day D14 produced a significant reversion of the subchronic PCP-induced deficit in the mouse T-maze alternation as indicated by the spontaneous alternation rate of 53%. The reversal effect of NX218 was even greater (62% spontaneous alternation) when PCP mice received three consecutive pretreatments (48 h, 24 h and 2 h) performed on D12, D13 and D14, respectively. In contrast, single 24 h pretreatment with NX218 was ineffective against the deficit as shown by a spontaneous alternation rate of 35%. Taken together, these results indicate that NX218 reversed subchronic PCP-induced cognitive deficits in mice but the presence of a 2 h pretreatment seems to be crucial.

Nicotine (0.4 mg/kg) tested in parallel produced a spontaneous alternation performance of 62%, significantly different from the subchronic PCP-treated mice.

### Cerebral Biomarkers Levels (Protein Expression Measured by Automated WB):

Effect of NX218 on the levels of relevant cerebral biomarkers in cortical samples of mice treated chronically with PCP are presented in tables 11 and 12 and in FIGS. 16 and 17.

TABLE 11
pCREB protein level in cortical samples in a mouse model of cognitive
deficits induced by the subchrnoic administrations of PCP (results
are expressed as a percentage of control condition)

| | | | PCP + | PCP + NX218 |
|---|---|---|---|---|
| | | PCP | NX218 | 5 mg/kg |
| | Control | D1-D11 | 5 mg/kg D14 | D12/D13/D14 |
| Mean | 100.00 | 44.17 | 68.67 | 102.22 |
| SEM | 13.94 | 4.98 | 7.91 | 11.40 |
| N | 5.00 | 5.00 | 5.00 | 5.00 |

TABLE 12
GluN2A level in cortical samples in a mouse model of cognitive
deficits induced by the subchronic administrations of PCP
(results are expressed as a percentage of control condition)

| | | | PCP + | PCP + NX218 |
|---|---|---|---|---|
| | | PCP | NX218 | 5 mg/kg |
| | Control | D1-D11 | 5 mg/kg D14 | D12/D13/D14 |
| Mean | 100 | 73.44 | 116.46 | 196.34 |
| SEM | 7.816044 | 7.66 | 6.47 | 41.19 |
| N | 5 | 5 | 5 | 5 |

The level of GluN2A was not modified in the cortices of PCP-treated mice when compared to control ones. However a significant increase in GluN2A total was observed in mice chronically treated with NX218 when compared to control mice (two-fold increase) and to PCP-treated mice. A significant reduction of the nuclear transcription factor pCREB was observed in the cortex of PCP-treated mice, that was restored after chronic administration of NX218.

Overall, the results indicate that NX218 can regulate the level and/or the phosphorylation of proteins involved in synaptic plasticity in the cortex.

### Conclusion

Finally, NX218 can reverse PCP-induced cognitive deficits in mice. Both repeated administrations of the peptide or a single acute administration of NX218 2 hours before the cognitive task robustly improved the cognitive performance of mice. The effect of repeated administrations of NX218 at synapses was transduced into an increase in GluN2A subunit cortical contents along with a restoration of CREB phosphorylation, two connected mechanisms that may explain the recovery of cognitive function.

It is shown herein that NX218 reinforced the strength of neurotransmission in different neural circuits (i.e., hippocampal or thalamocortical synapses), likely through increases in GluN2A-NMDAR and AMPAR excitatory postsynaptic currents. In a mouse model displaying defective synaptic transmission induced by the chronic administration of the NMDAR antagonist phencyclidine, a single acute systemic injection of NX218 robustly improved spatial working memory. Further, repeated daily treatments with NX218 increase GluN2A-NMDAR cortical contents meanwhile restoring NMDAR-dependent phosphorylation of CREB, thereby underpinning the full recovery of memory function.

AMPAR and NMDAR are main players of excitatory synaptic transmission whose persistent changes elicit plasticity through molecular cascades in various CNS areas to ensure essential functions such as learning and memory or neuroendocrine function. Mechanistically, the activation of AMPAR leads to a rapid depolarization of postsynaptic membranes which accelerates the electrical communication between neurons, whereas the activation of NMDAR regulates neuronal gene expression to maintain long-term changes induced by AMPAR. The diversity in AMPAR and NMDAR subunit composition and trafficking leads to many different forms of synaptic plasticity such as LTP and long-term depression (LTD) (critical for learning and memory processes), homeostatic plasticity or metaplasticity. In this study, we provide the first evidence that NX218 reinforces the strength of excitatory neurotransmission at CA3-CA1 hippocampal and thalamocortical synapses. NX218 thus represents a therapeutic opportunity to enhance excitatory neurotransmission in different disorders and states where the glutamatergic synaptic transmission is impaired such as schizophrenia or even normal aging. In addition, reduction of the glutamatergic system activity is often associated to a reduction of the GABAergic system activity to maintain balance between excitatory and inhibitory transmissions; the activity of both systems could therefore be enhanced by an exogenous supply of NX218. One major challenge in treating excitatory synaptic dysfunction is to reach a fast rebalancing of excitation and inhibition within the CNS without promoting excitotoxic neuronal death. Interestingly, we observed that GluN2A triggered the increase of EPSC in presence of NX218, a subunit known to promote both neurotransmission and neuronal survival.

As aforementioned, NMDAR play crucial roles in synaptic transmission and plasticity, and in cognitive processes. Accordingly, short- or long-term reduction of glutamate activity resulting from acute or chronic exposure to the NMDAR antagonist PCP impairs short-term spatial memory in rodents, primates and humans. In addition to its effect on excitatory currents at synapses and subsequent neurotransmission, NX218 also promotes NMDAR-driven signaling and plasticity, as shown by increases in both pCREB and GluN2A-NMDAR protein levels in presence of the peptide in vivo. Furthermore, we have provided evidence that the action of NX218 relieves mice from short-term memory deficits. We hypothesize that the effect of NX218 on synaptic plasticity and related functions (i.e., memory in this study) might be associated with postsynaptic increase in GluN2A-NMDAR protein levels rather that an increase in the presynaptic release in glutamate containing vesicles.

In summary, our study demonstrates that NX218 facilitates AMPAR- and GluN2A-NMDAR-mediated neurotransmission in brain areas associated with high-order functions (i.e., cortex and hippocampus). In line with these findings, we observed that NX218 treatment elicits favorable changes both in NMDAR-dependent signaling and in short-term memory in a pharmacological mouse model of synaptic dysfunction. Overall, the regulation of GluN2A-NMDAR and AMPAR function by NX218 represents an innovative therapeutic opportunity to ameliorate outcomes in the elderly and in patients suffering from CNS disorders with disabling synaptic defects.

### REFERENCES (INCORPORATED HEREIN BY REFERENCE)

Agmon A. & Connors B. W. (1991) Thalamocortical responses of mouse somatosensory (barrel) cortex in vitro. Neuroscience 41(2-3), 365-379. doi:10.1016/0306-4522(91)90333-j
Alnefeesi, Y. et al. (2020). Impact of SARS-CoV-2 Infection on Cognitive Function: A Systematic Review. Front Psychiatry, 11, 621773. doi: 10.3389/fpsyt.2020.621773
Álvarez Bravo, G., & Ramio I Torrenta, L. (2020). Anti-NMDA receptor encephalitis secondary to SARS-CoV-2 infection. Neurologia (Engl Ed), 35(9), 699-700. doi: 10.1016/j.nrl.2020.07.013
Bagnato, S. et al. (2013). Emerging from an unresponsive wakefulness syndrome: brain plasticity has to cross a threshold level. Neurosci Biobehav Rev, 37(10 Pt 2), 2721-2736. doi: 10.1016/j.neubiorev.2013.09.007
Balu, D. T. The NMDA Receptor and Schizophrenia: From Pathophysiology to Treatment. Adv. Pharmacol. 2016, 76, 351-382.
Benitez, D. P. et al. Knock-in models related to Alzheimer's disease: synaptic transmission, plaques and the role of microglia. Mol. Neurodegener. 2021, 16, 47.
Busse, S. et al. (2014). N-methyl-d-aspartate glutamate receptor (NMDA-R) antibodies in mild cognitive impairment and dementias. Neurosci Res, 85, 58-64. doi: 10.1016/j.neures.2014.06.002

Camporesi, E., Nilsson, J., Brinkmalm, A., Becker, B., Ashton, N. J., Blennow, K., & Zetterberg, H. (2020). Fluid Biomarkers for Synaptic Dysfunction and Loss. Biomarker insights, 15, 1177271920950319.
Chitty, K. M. et al. (2015). Hippocampal glutamatergic/NMDA receptor functioning in bipolar disorder: A study combining mismatch negativity and proton magnetic resonance spectroscopy. Psychiatry Res, 233(2), 88-94. doi: 10.1016/j.pscychresns.2015.05.002
Conway, M. E. Alzheimer's disease: targeting the glutamatergic system. Biogerontology 2020, 21, 257-274.
Cook, I. A., & Leuchter, A. F. (1996). Synaptic dysfunction in Alzheimer's disease: clinical assessment using quantitative EEG. Behavioural brain research, 78(1), 15-23.
Domino, E. F., & Luby, E. D. (2012). Phencyclidine/schizophrenia: one view toward the past, the other to the future. Schizophr Bull, 38(5), 914-919. doi: 10.1093/schbul/sbs011
Doss, S. et al. (2014). High prevalence of NMDA receptor IgA/IgM antibodies in different dementia types. Ann Clin Transl Neurol, 1(10), 822-832. doi: 10.1002/acn3.120
Farinelli M, Heitz F D, Grewe B F, Tyagarajan S K, Helmchen F and Mansuy I M (2012). Selective Regulation of NR2B by Protein Phosphatase-1 for the Control of the NMDA Receptor in Neuroprotection. PLoS ONE 7(3): e34047.
Finke, C. et al. (2012). Cognitive deficits following anti-NMDA receptor encephalitis. J Neurol Neurosurg Psychiatry, 83(2), 195-198. doi: 10.1136/jnnp-2011-300411
Finnema, S. J., et al. (2016). Imaging synaptic density in the living human brain. Science translational medicine, 8(348), 348ra96.
Garrido, M. I. et al. (2009). The mismatch negativity: a review of underlying mechanisms. Clin Neurophysiol, 120(3), 453-463. doi: 10.1016/j.clinph.2008.11.029
Gerlai, R. (1998) A new continuous alternation task in T-maze detects hippocampal dysfunction in mice. A strain comparison and lesion study. Behav Brain Res. 95(1), 91-101. doi: 10.1016/s0166-4328(97)00214-3.
Gilles, C., & Luthringer, R. (2007). Pharmacological models in healthy volunteers: their use in the clinical development of psychotropic drugs. J Psychopharmacol, 21(3), 272-282. doi: 10.1177/0269881107077733
Gould, T. J. (2010). Addiction and cognition. Addict Sci Clin Pract, 5(2), 4-14.
Hansen, K. B. et al. (2017). NMDA Receptors in the Central Nervous System. Methods Mol Biol, 1677, 1-80. doi: 10.1007/978-1-4939-7321-7_1
Hashimoto, K. (2014). Targeting of NMDA receptors in new treatments for schizophrenia. Expert Opin Ther Targets, 18(9), 1049-1063. doi: 10.1517/14728222.2014.934225
Hédou G F, Koshibu K, Farinelli M, Kilic E, Gee C E, Kilic U, Baumgsrtel K, Hermann D M and Mansuy I M (2008). Protein phosphatase 1-dependent bidirectional synaptic plasticity controls ischemic recovery in the adult brain. J Neurosci. 28(1):154-62.
Hiramatsu, M., & Inoue, K. (1999). Nociceptin/orphanin FQ and nocistatin on learning and memory impairment induced by scopolamine in mice. Br J Pharmacol, 127(3), 655-660. doi: 10.1038/sj.bjp.0702595
Hirotsu, I. et al. (1989). Effect of anticholinergic drug on long-term potentiation in rat hippocampal slices. Brain Res, 482(1), 194-197. doi: 10.1016/0006-8993(89)90561-1
Ingram, R. et al. (2018). Some distorted thoughts about ketamine as a psychedelic and a novel hypothesis based on NMDA receptor-mediated synaptic plasticity. Neuropharmacology, 142, 30-40. doi: 10.1016/j.neuropharm.2018.06.008
Itoh, J. et al. (1993). Dynorphin A-(1-13) markedly improves scopolamine-induced impairment of spontaneous alternation performance in mice. Eur J Pharmacol, 236(3), 341-345. doi: 10.1016/0014-2999(93)90469-x
Jaffe, D. B., & Brown, T. H. (1994). Metabotropic glutamate receptor activation induces calcium waves within hippocampal dendrites. J Neurophysiol, 72(1), 471-474. doi: 10.1152/jn.1994.72.1.471
Jedema, H. P. et al. (2021). Long-Term Cocaine Self-administration Produces Structural Brain Changes That Correlate With Altered Cognition. Biol Psychiatry, 89(4), 376-385. doi: 10.1016/j.biopsych.2020.08.008
Jones, C. A. et al. (2011). Animal models of schizophrenia. Br J Pharmacol, 164(4), 1162-1194. doi: 10.1111/j.1476-5381.2011.01386.x
Keralapurath, M. M. et al. (2017). Cocaine self-administration induces changes in synaptic transmission and plasticity in ventral hippocampus. Addict Biol, 22(2), 446-456. doi: 10.1111/adb.12345
Knobloch, M. et al. (2007). Abeta oligomer-mediated long-term potentiation impairment involves protein phosphatase 1-dependent mechanisms. J Neurosci, 27(29), 7648-7653. doi: 10.1523/JNEUROSCI.0395-07.2007
Koch, J. M. et al. (2009). Changes in CREB phosphorylation and BDNF plasma levels during psychotherapy of depression. Psychotherapy and psychosomatics, 78(3), 187-192.
Kumar, A.; Thinschmidt, J. S.; Foster, T. C. Subunit contribution to NMDA receptor hypofunction and redox sensitivity of hippocampal synaptic transmission during aging. Aging (Albany NY) 2019, 11, 5140-5157.
Kumar, S. et al. (2021). Neuropsychiatric and Cognitive Sequelae of COVID-19. Front Psychol, 12, 577529. doi: 10.3389/fpsyg.2021.577529
Lepeta, K. et al. Synaptopathies: synaptic dysfunction in neurological disorders—A review from students to students. J. Neurochem. 2016, 138, 785-805.
Luo, Y. et al. (2021). Chronic administration of ketamine induces cognitive deterioration by restraining synaptic signaling. Mol Psychiatry. doi: 10.1038/s41380-020-0793-6
Maurice, T. et al. (1996). Amnesia induced in mice by centrally administered beta-amyloid peptides involves cholinergic dysfunction. Brain Res, 706(2), 181-193. doi: 10.1016/0006-8993(95)01032-7
Maurice, T. et al. (1998). Sigma1 (sigma 1) receptor agonists and neurosteroids attenuate B25-35-amyloid peptide-induced amnesia in mice through a common mechanism. Neuroscience, 83(2), 413-428. doi: 10.1016/s0306-4522(97)00405-3
Medina-Kirchner, C., & Evans, V. (2021). Cognitive impairment in ketamine abusers. Psychiatry Res, 296, 113589. doi: 10.1016/j.psychres.2020.113589
Meunier, J. et al. (2006). The anti-amnesic and neuroprotective effects of donepezil against amyloid beta25-35 peptide-induced toxicity in mice involve an interaction with the sigma1 receptor. Br J Pharmacol, 149(8), 998-1012. doi: 10.1038/sj.bjp.0706927
Milnerwood, A. J. et al. Early synaptic pathophysiology in neurodegeneration: insights from Huntington's disease. Trends Neurosci. 2010, 33, 513-523.
More, S. V. et al. (2016). Toxin-Induced Experimental Models of Learning and Memory Impairment. Int J Mol Sci, 17(9). doi: 10.3390/ijms17091447
Nakazawa, K.; Sapkota, K. The origin of NMDA receptor hypofunction in schizophrenia. Pharmacol. Ther. 2020, 205, 107426.
Neill, J. C. et al. (2010). Animal models of cognitive dysfunction and negative symptoms of schizophrenia: focus on NMDA receptor antagonism. Pharmacol Ther, 128(3), 419-432. doi: 10.1016/j.pharmthera.2010.07.004Olam N & Ibiloglu A O. (2019). Changes in CREB Activity and BDNF Plasma Levels in Patients with Post-Traumatic Stress Disorder (PTSD). Psychiatry and Behavioral Sciences, 9(4):158-165.
Pearlman, D. M., & Najjar, S. (2014). Meta-analysis of the association between N-methyl-d-aspartate receptor antibodies and schizophrenia, schizoaffective disorder, bipolar disorder, and major depressive disorder. Schizophr Res, 157(1-3), 249-258. doi: 10.1016/j.schres.2014.05.001
Pistoia, F. et al. (2010). Awakenings and awareness recovery in disorders of consciousness: is there a role for drugs?CNS Drugs, 24(8), 625-638. doi: 10.2165/11535940-000000000-00000
Pons-Bennaceur, A., & Lozovaya, N. (2017). Electrophysiological Investigation of NMDA Current Properties in Brain Slices. Methods Mol Biol, 1677, 231-239. doi: 10.1007/978-1-4939-7321-7_12
Pratt, J. et al. (2017). Thalamo-cortical communication, glutamatergic neurotransmission and neural oscillations: A unique window into the origins of ScZ? Schizophr Res, 180, 4-12. doi: 10.1016/j.schres.2016.05.013
Pu, L. et al. (2002). Hippocampal long-term potentiation is reduced by chronic opiate treatment and can be restored by re-exposure to opiates. J Neurosci, 22(5), 1914-1921.
Ren, X. et al. (2014). Alteration of cyclic-AMP response element binding protein in the postmortem brain of subjects with bipolar disorder and schizophrenia. Journal of affective disorders, 152-154, 326-333
Roussy, M. et al. (2021). Ketamine disrupts naturalistic coding of working memory in primate lateral prefrontal cortex networks. Mol Psychiatry. doi: 10.1038/s41380-021-01082-5
Sarigecili, E. et al. (2021). Pediatric anti-NMDA receptor encephalitis associated with COVID-19. Childs Nerv Syst. doi: 10.1007/s00381-021-05155-2
Scarr, E. et al. (2003). Decreased hippocampal NMDA, but not kainate or AMPA receptors in bipolar disorder. Bipolar Disord, 5(4), 257-264. doi: 10.1034/j.1399-5618.2003.00024.x
Sharma V K, Singh T G. CREB: A Multifaceted Target for Alzheimer's Disease. Curr Alzheimer Res. 2020; 17(14):1280-1293.
Shi, L.; Adams et al. J. K. Subtle alterations in glutamatergic synapses underlie the aging-related decline in hippocampal function. In Brain Aging: Models, Methods, and Mechanisms, Riddle, D. R; Boca Raton (FL): CRC Press/Taylor & Francis, 2007; Chapter 8.
Silva A. J., Kogan J. H., Frankland P. W., Kida S., CREB and memory, Annu. Rev. Neurosci., 1998, vol. 21 (pg. 127-148)
Stanca, D. M. et al. (2015). GFAP and antibodies against NMDA receptor subunit NR2 as biomarkers for acute cerebrovascular diseases. J Cell Mol Med, 19(9), 2253-2261. doi: 10.1111/jcmm.12614
Stringer, J. L. et al. (1983). Blockade of long-term potentiation by phencyclidine and sigma opiates in the hippocampus in vivo and in vitro. Brain Res, 280(1), 127-138. doi: 10.1016/0006-8993(83)91180-0.
Stringer, J. L., & Guyenet, P. G. (1983). Elimination of long-term potentiation in the hippocampus by phencyclidine and ketamine. Brain Res, 258(1), 159-164. doi: 10.1016/0006-8993(83)91244-1.
Stuart, G. J., & Sakmann, B. (1994). Active propagation of somatic action potentials into neocortical pyramidal cell dendrites. Nature, 367(6458), 69-72. doi: 10.1038/367069a0.
Stuart, G. J. et al. (1993). Patch-clamp recordings from the soma and dendrites of neurons in brain slices using infrared video microscopy. Pflugers Arch, 423(5-6), 511-518. doi: 10.1007/BF00374949.
Tang, K. S. (2019). The cellular and molecular processes associated with scopolamine-induced memory deficit: A model of Alzheimer's biomarkers. Life Sci, 233, 116695. doi: 10.1016/j.lfs.2019.116695.
Tang, W. et al. (2020). Positive allosteric modulators that target NMDA receptors rectify loss-of-function GRIN variants associated with neurological and neuropsychiatric disorders. Neuropharmacology, 177, 108247. doi: 10.1016/j.neuropharm.2020.108247.
Tzang, R. F. et al. (2019). Autism Associated With Anti-NMDAR Encephalitis: Glutamate-Related Therapy. Front Psychiatry, 10, 440. doi: 10.3389/fpsyt.2019.00440.
Varela, C. (2013). The gating of neocortical information by modulators. J Neurophysiol, 109(5), 1229-1232. doi: 10.1152/jn.00701.2012.
Villard, V. et al. (2009). Antiamnesic and neuroprotective effects of the aminotetrahydrofuran derivative ANAVEX1-41 against amyloid beta(25-35)-induced toxicity in mice. Neuropsychopharmacology, 34(6), 1552-1566. doi: 10.1038/npp.2008.212.
Villard, V. et al. (2011). Anti-amnesic and neuroprotective potentials of the mixed muscarinic receptor/sigma 1 (σ1) ligand ANAVEX2-73, a novel aminotetrahydrofuran derivative. J Psychopharmacol, 25(8), 1101-1117. doi: 10.1177/0269881110379286.
Wagnon, I. et al. (2020). Autoimmune encephalitis mediated by B-cell response against N-methyl-d-aspartate receptor. Brain, 143(10), 2957-2972. doi: 10.1093/brain/awaa250.
Wang, H., Xu, J., Lazarovici, P., Quirion, R., & Zheng, W. (2018). cAMP Response Element-Binding Protein (CREB): A Possible Signaling Molecule Link in the Pathophysiology of Schizophrenia. Frontiers in molecular neuroscience, 11, 255.
Yang, A. C. et al. (2021). Dysregulation of brain and choroid plexus cell Itypes in severe COVID-19. Nature, doi: 10.1038/s41586-021-03710-0.
Young, J. W. et al. (2012). Mouse pharmacological models of cognitive disruption relevant to schizophrenia. Neuropharmacology, 62(3), 1381-1390. doi: 10.1016/j.neuropharm.2011.06.013.
Yuan, P. et al. (2010). Altered levels of extracellular signal-regulated kinase signaling proteins in postmortem frontal cortex of individuals with mood disorders and schizophrenia. Journal of affective disorders, 124(1-2), 164-169.

1-14. (canceled)

15. A method of treating a subject in need thereof to enhance or restore GluN2A-NMDAr-mediated glutamatergic neurotransmission, the method comprising administering to the subject a therapeutic amount of a peptide and a pharmaceutically acceptable vehicle or excipient, wherein said peptide has the following amino acid sequence
X1-W-S-A1-W-S-A2-C-S-A3-A4-C-G-X2
in which:
A1, A2, A3 and A4 consists of amino acid sequences consisting of 1 to 5 amino acids,
X1 and X2 consists of amino acid sequences consisting of 1 to 6 amino acids; or
X1 and X2 are absent;
it being possible for the N-terminal amino acid to be acetylated, for the C-terminal amino acid to be amidated, or the N-terminal amino acid to be acetylated and the C-terminal amino acid to be amidated.

16. The method of claim 15, which is for treating said subject to enhance or restore AMPAr and GluN2A-NMDAr-mediated glutamatergic neurotransmission.

17. The method of claim 15, which is for treating said subject to enhance or restore excitatory synaptic transmission when this transmission is compromised.

18. The method of claim 17, wherein this transmission is compromised during or after hypoxia.

19. The method of claim 15, which is for treating said subject to treat or prevent Schizophrenia, drug addiction, anti-NMDAr encephalitis, hypoxia-induced depression of synaptic transmission and/or hypoxic brain injury, synaptic deficits resulting from a viral infection, synaptic dysfunction of a synaptopathy, or to treat bipolar disorder or vegetative state.

20. The method of claim 15, which is for treating said subject to treat or prevent synaptic deficits resulting from SARS CoV2 or COVID-19.

21. The method of claim 15, wherein the peptide is of amino acid sequence W-S-A1-W-S-A2-C-S-A3-A4-C-G, in which A1, A2, A3 and A4 consists of amino acid sequences consisting of 1 to 5 amino acids.

22. The method of claim 15, wherein
A1 is chosen from G, V, S, P and A,
A2 is chosen from G, V, S, P and A,
A3 is chosen from R, A and V, and/or
A4 is chosen from S, T, P and A.

23. The method of claim 15, wherein A1 and A2 are independently chosen from G and S, and/or A3-A4 is chosen from R-S or V-S or V-T or R-T.

24. The method of claim 15, wherein the peptide is of a sequence selected from the group consisting of sequences SEQ ID NO: 3-63.

25. The method of claim 15, wherein the peptide is a linear peptide or a cyclized peptide wherein the two cysteines form a disulfide bridge.

26. The method of claim 15, which is the peptide of sequence SEQ ID NO: 3, wherein the peptide is a linear peptide or a cyclized peptide wherein the two cysteines form a disulfide bridge, or a mixture of both.

27. The method of claim 15, which is the peptide of sequence SEQ ID NO: 3, wherein the peptide is a cyclized peptide wherein the two cysteines form a disulfide bridge.

28. The method of claim 15, wherein the peptide increases, enhances or restores excitatory synaptic transmission in said subject.

29. The method of claim 15, wherein the peptide prevents or treats the deleterious effect of hypoxia on excitatory synaptic transmission in said subject.

30. The method of claim 15, which is for treating said subject to increase, enhance or restore excitatory synaptic transmission.

31. The method of claim 15, which is for treating said subject to increase, enhance or restore excitatory synaptic transmission in the hippocampus.

32. The method of claim 15, which is for treating said subject to prevent or treat the deleterious effect of hypoxia on excitatory synaptic transmission.

33. The method of claim 15, which is for treating said subject to prevent or treat the deleterious effect of hypoxia on excitatory synaptic transmission in the hippocampus.

34. A method of treating a subject in need thereof to treat or prevent Schizophrenia, drug addiction, anti-NMDAr encephalitis, hypoxia-induced depression of synaptic transmission and/or hypoxic brain injury, synaptic deficits resulting from a viral infection, synaptic dysfunction of a synaptopathy, or to treat bipolar disorder or vegetative state, the method comprising administering to the subject a therapeutic amount of a peptide and a pharmaceutically acceptable vehicle or excipient, wherein said peptide has the following amino acid sequence
X1-W-S-A1-W-S-A2-C-S-A3-A4-C-G-X2
in which:
A1, A2, A3 and A4 consists of amino acid sequences consisting of 1 to 5 amino acids,
X1 and X2 consists of amino acid sequences consisting of 1 to 6 amino acids; or
X1 and X2 are absent;
it being possible for the N-terminal amino acid to be acetylated, for the C-terminal amino acid to be amidated, or the N-terminal amino acid to be acetylated and the C-terminal amino acid to be amidated.
